(19) United States
(12) Patent Application Publication
(10) Pub. No.: US 2014/0155910 A1
(43) Pub. Date: Jun. 5, 2014

(54) Spherical Motion Mechanism

(71) Applicant: University of Washington Through Its Center for Commercialization, Seattle, WA (US)

(72) Inventors: Blake Hannaford, Seattle, WA (US); Jacob Rosen, Santa Cruz, CA (US); Dennis Trimble, Seattle, WA (US); Mitchell Lum, Seattle, WA (US); Manuel Moreyra, Shoreline, WA (US); Mika Sinanan, Brier, WA (US)

(73) Assignee: University of Washington Through Its Center for Commercialization, Seattle, WA (US)

(21) Appl. No.: 13/908,120

(22) Filed: Jun. 3, 2013

Related U.S. Application Data
(63) Continuation of application No. 12/825,236, filed on Jun. 28, 2010.
Application No. 12/825,236 is a continuation-in-part of application No. 11/113,824, filed on Apr. 25, 2005, now abandoned.
Application No. 11/113,824 is a continuation-in-part of application No. 11/466,269, filed on Aug. 22, 2006, now abandoned.
(60) Provisional application No. 60/711,514, filed on Aug. 26, 2005.

(51) Int. Cl. A61B 19/00

(52) U.S. Cl. CPC A61B 19/22; A61B 19/2203
USPC 606/130; 29/428; 74/89; 74/89.2

(57) ABSTRACT

This document discusses, among other things, a mechanism for providing motion having two degrees of freedom and centered on a single point within a sphere. Methods to design the mechanism consistent with specified parameters are also described.

### CROSS-REFERENCES TO RELATED APPLICATIONS

This application is a continuation of a copending U.S. patent application Ser. No. 12/825,236, entitled “Skill Evaluation Using Spherical Motion Mechanism,” filed on Jun. 28, 2010, which is a continuation-in-part of U.S. patent application Ser. No. 11/113,824, entitled “Spherical Motion Mechanism,” filed on Apr. 25, 2005, and also a continuation-in-part of U.S. patent application Ser. No. 11/466,269, entitled “Skill Evaluation,” filed on Aug. 22, 2006, which claims the benefit of priority, under 35 U.S.C. Section 119(e) to U.S. provisional patent application 60/711,514, entitled “Skill Evaluation,” filed on Aug. 26, 2005, the full disclosures of which are incorporated by references in their entirety.

### FEDERAL FUNDING STATEMENT

The invention was made with Government support under grant number DAMD17-97-1-7256 awarded by the Defense Advanced Research Projects Agency (DARPA); under grant number W81XWH-041-0464 awarded by the Department of Defense (DOD); under an Information Technology Research (ITR) award from the National Science Foundation (NSF); and under contract or grant number DAMD17-02-1-0202 awarded by the Department of the Army. The U.S. Government has certain rights in this invention.

### TECHNICAL FIELD

This document pertains generally to mechanical linkages, and more particularly, but not by way of limitation, to a spherical motion mechanism. This document also pertains generally to evaluations, and more particularly, but not by way of limitation, to skill evaluation.

### BACKGROUND OF THE INVENTION

Robotically controlled surgical methods, such as laparoscopy, hold the promise of bringing advanced medical procedures to the battlefield and other remote locations throughout the globe. Typical systems are often too large, heavy and cumbersome to be effective in an operating room. In addition, typical systems are subject to device collisions.

Human performance of a task, such as surgery, is evaluated for various reasons, including for example, developing skills and identifying expertise. Objective and subjective evaluation criteria can be established for evaluating or judging the performance of a subject. Some examples of tasks in which a subject uses physical controls to manipulate a mechanism include surgery, driving a vehicle, and operating machinery.

Typical methods of evaluating performance entail human oversight and are, thus, financially burdensome and often imprecise.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 includes a diagram showing selected modalities for surgery;

FIG. 2 includes a table of definitions for 15 states based on a spherical coordinate system;

FIG. 3 illustrates time charts for left and right endoscopic tools of a surgical robot system during a surgical procedure;

FIG. 4 illustrates vector representation of exemplary data;

FIG. 5 illustrates an exemplary cluster center;

FIG. 6 illustrates selected degrees of freedom;

FIGS. 7A and 7B illustrate a finite state diagram;

FIG. 8 illustrates exemplary Markov models represented as coded probabilistic maps;

FIG. 9 schematically illustrates statistical distances relative to an expert;

FIG. 10 illustrates normalized Markov model-based statistical distances;

FIG. 11 includes a perspective view of a serial version of a spherical motion mechanism;

FIG. 12 includes a side view of a spherical motion mechanism;

FIG. 13 includes a motor driven spherical motion mechanism with a linear actuator;

FIG. 14 includes a parallel version of a spherical motion mechanism; and

FIG. 15 includes a flow chart of a method for selecting a design of a spherical motion mechanism.

### DETAILED DESCRIPTION OF THE INVENTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

Figures and disclosed embodiments are not limiting. Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein. Further, it should be understood that any feature of one embodiment disclosed herein can be combined with one or more features of any other embodiment that is disclosed, unless otherwise indicated.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

### Overview

The present subject matter includes methods and systems for evaluating skills. Exemplary methods utilize a Markov model or hidden Markov model for analyzing the departure of a specific signal from what is expected by that model.

The present subject matter is described in this document largely based on Markov and hidden Markov models. Nevertheless, other types of models are also contemplated, including algorithmic or rule-based models, dynamical system models and statistical models (of which Markov and hidden Markov models are but two examples).

In one example, the performances of surgical skills on a pig by several participants were recorded and a model based on data generated from experts performing the skills has been created. The present subject matter distinguishes between signals generated by experts and non-experts and can be applied to non-surgical manipulative tasks including human or non-human operation of a machine. For example, the present subject matter can facilitate analysis of manipulations of physical controls used to operate a mechanism, such as driving a vehicle (steering wheel and pedals), flying an aircraft (yoke and pedals), operating machinery (such as a crane) and minimally invasive surgery.

Markov and hidden Markov models are exemplary statistical models which can be used for voice recognition of speech. Models of speech sounds are created in a controlled manner and a sample sound is recognized based on a comparison of the sample sound with those models. Statistical models, such as Markov and hidden Markov models, can tolerate variations in utterance of a particular word.

In the present subject matter, electrical signals derived from surgical instruments are used as a source input. The electrical signals are generated by sensors coupled to a surgical instrument when manipulated by operators performing at various skill levels. Surgical skill models are developed based on the recorded information. Once trained, data recorded by other surgeons (including experts and novices) are examined using the model. The model can be used to identify expert surgeons in a group. In one example, the present subject matter includes a skill measurement tool.

The analysis of the data recorded during surgery can be done off-line. That is, data analysis (and expert identification) is conducted after completion of the surgical procedure.

In one example, the data analysis is conducted in real time. That is, data processing and quantification of the skill level of subjects is performed concurrent with data acquisition.

In one example, large amounts of recorded data is compressed and simplified using vector quantization. Vector quantization was initially developed for image compression and it is adapted for use in the present subject matter.

The method includes receiving electric signals associated with a subject performing a particular task. Greater number of signals provides improved performance. In one example, the method includes receiving data recorded by experts to train a model.

In one example, a surgical robot is used to train subjects and subject performance evaluation is generated in real time. Feedback provided by the present system can augment skill development and reduce the burden of supervision.

In one example, a robotically controlled interface is coupled to one or more simulators for training purposes.

In one example, subjects are scored on their performance based on a simulated or actual manipulative task. In one example, performance is evaluated using a simulation prior to performing an actual complex procedure. Feedback derived from the evaluated simulation can be used to tailor actual performance. For example, surgeon performance using a surgical simulator can be evaluated prior to conducting actual surgery on a patient. The evaluation may reveal that the subject's performance is inferior to that of an expert because of fatigue or other correctable factor.

In one example, an interface includes a layer operating in the background of the surgical environment (actual, virtual or robotically controlled) which can interject upon detection of a departure from an expert performance. For example, if the conduct of a lower skilled surgeon is detected, then at a critical procedure, the layer will interrupt and prevent harmful movement or interrupt and suggest an improved course or provide tactile feedback (haptic) sensations to cause the surgeon to alter their performance. The layer can be implemented in hardware or in instructions executed by a computer of the present subject matter. In one example, the background layer fulfills a supervisory role as to a manipulative task.

The Markov decision process makes decisions by prioritizing possible choice as measured by evolving values criteria.

Assessing Skill with Medical Simulators

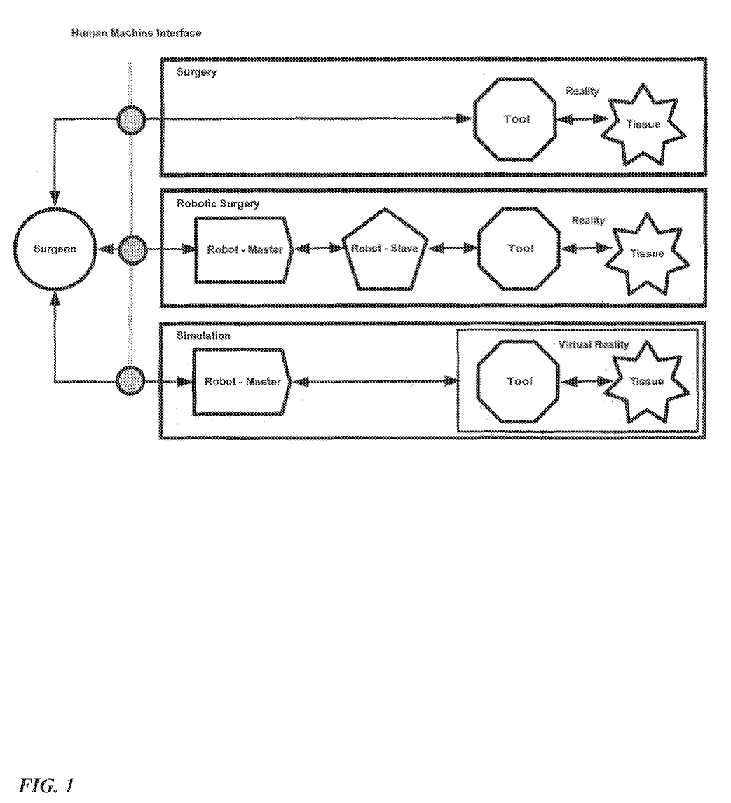

In the surgical context, procedurally-oriented skills can be performed utilizing three different modalities, (a) during actual open or minimally invasive clinical procedures; (b) in physical or virtual reality simulators with or without haptic feedback; and (c) during interaction with surgical robotic systems, as shown in FIG. 1. During open or minimally invasive surgical (MIS) procedures, the surgeon interacts with the patient's tissue either directly with his/her hands or through the mediations of tools. Surgical robotics enables the surgeon to operate in a tele-operation mode with or without force feedback using a master/slave system configuration. In this mode of operation, visualization is obtained from either an external camera or an endoscopic camera. Incorporating force feedback, allows the surgeon to feel through the master console the forces being applied on the tissue by the surgical robot, the slave, as he/she interacts with it from the master console. For training in a simulated virtual environment, the surgical tools, the robot-slave, and the anatomical structures are replaced with virtual counterparts. The surgeon interacts with specially-designed input devices, haptic devices when force feedback is incorporated, that emulate surgical tools, or with the master console of the robotic system itself, and performs surgical procedures in virtual reality.

In each modality, the surgeon is separated from the treated tissue or medium by an instrument or a mechanical interface. In some examples, the interface includes a virtual component. The intermediate modality in all these examples can be considered interchangeable. A common element of these modalities is the human-machine interface in which visual, kinematics, dynamic, and haptic information is shared between the surgeon and the various modalities. This interface can provide multi-dimensional data to objectively assess technical surgical skill within the general framework of surgical ability.

The algorithm used for objective assessment of skill is independent of the modality actually used and therefore, the same algorithms can be incorporated into any of these technologies. Objective methodologies for assessing task or skill competence and performance can be used to enhance training, reduce cost and improve competency.

In one example, the surgical task is deconstructed or decomposed to expose and analyze the internal hierarchy of tasks. Task decomposition is associated with defining selected elements of the manipulative process. For example, in surgery, the procedure is divided into steps, stages, or phases with defined intermediate goals. Additional hierarchical decomposition is based on identifying tasks or subtasks and actions or states. Low-level elements of the task decomposition are associated with quantify measurable parameters. Definition of these states along with measurable, quantitative data allows for modeling of surgical tasks or medical examination.

The present subject matter can be applied to the various modalities and includes decomposing the medical procedure (such as an examination or surgical task) into fundamental states associated with discrete observations. The task is represented by a statistical model such as a multi-state Markov model, a hidden Markov model, or other such model. A performance of a test subject is evaluated based on the statistical distance calculated between the test subject and at least one stored model. In one example, the stored models correspond to performance of the task at various skill levels, including that of a novice and an expert. The analysis can be conducted in real-time and provide feedback during the performance. Feedback, in various examples, can be in the form of audio, visual, or tactile. The present subject matter can be used with various modalities and systems (including robotic systems and simulators) for evaluating performance of a manipulative task.

In the present subject matter, a prime element is modeled by a finite state. In the context of Markov modeling and speech recognition, the prime element is the spoken word. The prime element in the surgical context relates to tool-tissue interaction or hand-tissue interaction. Within a particular tool-tissue interaction or hand-tissue interaction, variations in forces and torque magnitudes can be noted for different skill levels and, in the context of speech recognition, this relates to variations in word pronunciation. The various force and torque magnitudes are simulated by discrete observations in the model. A sequence of tool-tissue or hand-tissue interactions comprise the steps of a medical procedure having intermediate and specific outcomes, and by analogy in the speech recognition context, a sequence of words represent a sentence or chapter.

A variety of sensors are used to generate signals corresponding to, for example, completion time, work space, force, position, and tool path.

### Example

In one example, a physical simulator in the form of an instrumented teaching-mannequin representing the female pelvis and the breast exam, male prostate exam, and endotracheal intubation was used. Data was acquired from approximately 1800 students and clinicians, including quantitative measures of hands-on clinical exam techniques used while performing procedures. Background information for the students and clinicians, and a database of outcome measures including the user's clinical assessment scores and independent skilled observer ratings of the users' techniques while performing these examinations or procedures in physical simulators, was also collected.

Sensors coupled to surgical robotic systems were used to collect data on surgical tool positions and the torque commands between the master unit and the robotic instrument actuators.

Markov modeling, according to the present subject matter, provides an objective assessment of medical/surgical skills in a manner transparent to modality.

In one example, data mining is performed on a database corresponding to a manipulative task. A surgical robot provides data generated by sensors while performing surgical tasks on animal and human subjects.

In one example, two-handed, instrumented endoscopic tools and Markov models are used to perform task decomposition and objective skill assessment with the Markov modeling approach. Sensor arrays coupled to the tools and robotic systems provide quantitative data to allow data mining and clustering and multi-state Markov modeling and analysis of the particular tasks.

Objective assessment of surgical competence during minimally invasive surgery procedures is a multi-dimensional problem. Minimally invasive surgery (MIS) refers to a surgical procedure involving a minimally invasive surgical setup. Physiological constraints (stress, fatigue), equipment constraints (camera rotation and port location), team constraints (nurses), and physician ability are representative parameters that affect the outcome of a MIS procedure. Ability, with respect to surgery, is defined as the natural state or condition of being capable; innate aptitude (prior to training), which an individual brings for performing a surgical task. Minimally invasive surgery ability includes cognitive factors (knowledge and judgment) and technical factors (psychomotor ability, visio-spatial ability and perceptual ability). By definition, fundamental psychometric abilities are fixed at birth or early childhood and show little or no learning effect. However training enables the subject to perform as close as possible to his or her inherent psychometric abilities.

The methodology for objectively assessing surgical skill (as a subset of surgical ability), according to the present subject matter, includes objective and quantitative analysis. Such methodology is enabled by using instrumented tools, measurements of the surgeon's arm kinematics, gaze patterns, physical simulators, a variety of virtual reality simulators (those with and without haptics), and robotic systems. An instrumented tool can be used to generate data corresponding to kinematics (position, velocity, acceleration, and jerk), dynamics (force, and torque), contact information between the tool and the medium (e.g., real tissue or simulated tissue), and recorded display of the scene in the proximity of the tool.

Regardless of the modality being used or the clinical procedure being studied, task deconstruction or decomposition is one component of an objective skills-assessment methodology. Exposing and analyzing the internal hierarchy of tasks provides an objective means for quantifying training and skills acquisition.

Task decomposition is associated with defining the prime elements of the manipulative task. In surgery, a particular procedure is divided into steps, stages, or phases with well-defined intermediate goals. Additional hierarchical decomposition is based upon identifying tasks or subtasks including a sequence of actions or states. In addition, other measurable parameters such as workspace completion time, tool position, and forces and torques can be analyzed. Selecting low-level elements of the task decomposition allows one to associate these elements with quantifiable and measurable parameters. The definition of these states, along with measurable, quantitative data, are used for modeling and examining surgical tasks as a process.

In the proposed study, an analogy between minimally invasive surgery (MIS) and the human language inspires the decomposition of a surgical task into its prime elements. Modeling the sequential element expressions using a multi-finite states model (for example, a Markov model) reveals the internal structure of the surgical task which is utilized in assessing surgical performance. Markov modeling (MM) and hidden Markov modeling (HMM), a subset of MM, are used to characterize manipulative tasks.

Within the context of the three modalities (direct surgery/clinical examination, simulated procedures—either physical or virtual, and surgical robot), the procedure can be summarized as follows:

(a) decompose the clinical task into fundamental states associated with discrete events (observations);

(b) represent the task using a statistical model such as a multi-state Markov model; and

(c) determine statistical distances between a subject performance and models representing subjects with various skill levels.

In one example, the present subject matter includes procedures for analyzing a database acquired from two modalities (simulator and instrumented surgical tools) using vector quantization algorithms.

According to one example, a method includes decomposing the task using expert knowledge and developing the Markov model architectures, training the Markov models based on the processed data, developing the learning curves based on measuring the statistical similarity between the models representing subjects at different levels of surgical training to enable an objective assessment of surgical skills and generalizing the methodology for assessing skill in the three modalities.

In the context of battlefield conditions, for example, military medical personnel may be called upon to perform tasks that may exceed the complexity or skill of civilian medical personnel. Even extended experience in a civilian trauma center may be inadequate to prepare military personnel to perform under realistic conditions. As such, simulators are valuable tools in training military personnel. In addition, a mechanism for assessing skill can be helpful in a simulator and in particular, a simulator used to train military medical care providers.

Among other applications, a statistical model, such as a Markov model, can provide a tool in developing a methodology for studying models of the human operator in complex interactive tasks with machines.

### Databases and Data Collection

A particular surgical robot, known popularly as the BlueDRAGON, is a system developed at the University of Washington for acquiring the kinematics and the dynamics of two endoscopic tools along with the visual view of the surgical scene while performing a MIS procedure. The system includes two four-bar passive mechanisms attached to two endoscopic tools. During a minimally invasive surgical procedure, the endoscopic tool is inserted into the body through a port located, for example, in the abdominal wall. The tool is rotated around a pivot point within the port that is generally inaccessible for sensors aimed to measure rotation of the tool. The position and orientation of the tool, with respect to the port, is tracked by sensors that are incorporated into the joints of the mechanism. The two mechanisms are equipped with three classes of sensors.

A first class of sensors include position sensors (such as potentiometers) incorporated into four of the joints of the mechanisms for measuring the position, orientation and translation of the two instrumented endoscopic tools attached thereto. In addition, two linear potentiometers are attached to the handles of the tools and used for measuring the endoscopic handle and tool tip angles.

A second class of sensors include three-axis force/torque (F/T) sensors (with holes drilled at their center) that are inserted and clamped to the proximal end of the shafts of the endoscopic tools. In addition, double beam force sensors are inserted into the handles of the tools for measuring the grasping forces at the hand-tool interface.

A third class of sensors include contact sensors, based on a resistance-capacitance (RC) circuit, which provides a binary indication of tool-tip/tissue contact.

Data measured by the sensors are acquired using two 12-bit USB A/D cards sampling the 26 channels (4 rotations, 1 translation, 1 tissue contact, and 7 channels of forces and torques from each instrumented grasper) at a frequency of 30 Hz. In addition to data acquisition, the synchronized view of the surgical scene is incorporated into a graphical user interface displaying data in real-time.

Preliminary tests acquiring data at a sampling rate of 1 KHz indicated that 95% of the signals' accumulated energy is in a bandwidth 0-5 Hz. In addition, a graphical user interface (GUI) is provided to display information measured by the surgical robot in real-time while incorporating endoscopic view of the surgical scene acquired by the endoscopes video camera. On the top right side of the GUI, a virtual representation of the two endoscopic tools are shown along with vectors representing the instantaneous velocities. On the bottom left a three dimensional representation of the forces and torque vectors are presented. Surrounding the endoscopic image are bars representing the grasping/spreading forces applied on the handle and transmitted to the tool tip via the tool's internal mechanism, along with virtual binary LED indicating contact between the tool tips and the tissues.

A representative physical simulator is popularly known as the E-Pelvis. The E-pelvis is a physical simulator developed at Stanford University that consists of a partial mannequin (umbilicus to mid-thigh) constructed in the likeness of an adult human female. The mannequin is instrumented internally with force sensors that are connected to a computer having a graphical user interface for providing a real-time visual feedback. Test subjects perform simulated clinical female pelvic examinations on the mannequin and the data is collected at a sampling frequency of 30 Hz and stored in a memory for off-line analysis.

A representative surgical robot system, popularly known as DaVinci, is commercially available from Intuitive Surgical (Sunnyvale, Calif.) and is FDA approved for selected surgical procedures. The system is equipped with an interface card that allows passive acquisition of internal variables of the robot during operation. Examples of data generated include position of the surgical tools and motor commands. The data is sampled at 30 Hz, displayed in real time by using a user interface and stored for off-line analysis.

### Protocol for the Surgical Robot

The protocol using the surgical robot included collecting data from task performances conducted by surgeons having different levels of expertise. In one example, the performances of 30 surgeons were monitored. Levels of expertise ranged from surgeons in training to surgical attending physicians. Five subjects in each group represented the five years of surgical training, (5×R1, R2, R3, R4, R5—where the numeral denotes year of training) and five expert surgeons. For the purpose of this example, an expert surgeon (E) was defined as a board certified laparoscopic surgeon who performed at least 800 surgeries and practices medicine as an attending physician. Each subject was given instruction through a multimedia presentation on how to perform three basic surgical tasks involving (1) tying an intracorporeal knot; (2) manipulating tissue; and (3) tissue dissection. The multimedia presentation included a written description of the task and a video clip of the surgical scene with audio explanation of the task. Subjects were then given 15 minutes in which to complete this task in a swine model.

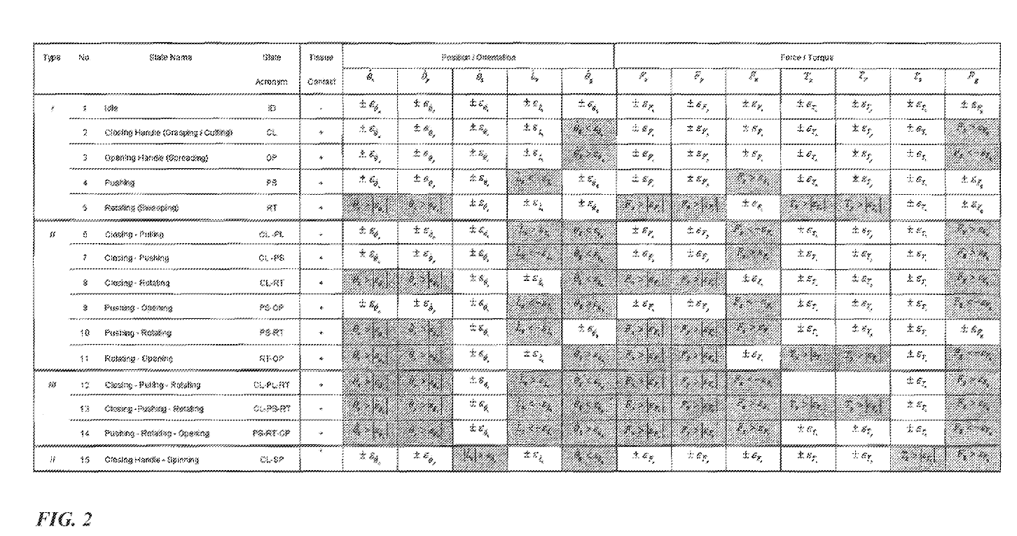

In addition to the surgical task, each subject performed 15 predefined tool/tissue and tool/needle-suture interactions as shown in FIG. 2. The definitions of the 15 states are based on a spherical coordinate system with an origin at the port. Each state features a unique set of angular/linear velocities, forces and torques. A non-zero threshold value is defined for each parameter by ε. The states' definitions are independent from the tool tip being used. For example, the state defined as Closing Handle might be associated with grasping or cutting if a grasper or scissors are being used respectively.

The kinematics (that is, the position/orientation (P/O) of the tools in space with respect to the port), and the dynamics (that is, forces and torque—F/T—applied by the surgeons on the tools) of the left and right endoscopic tools along with the visual view of the surgical scene were acquired by a passive mechanism coupled to the surgical robot. This data provided the F/T and velocity signatures associated with each interaction that were then used as the model observations associated with each state of the model.

### Protocol for the Physical Simulator

The experimental protocol for the simulator included 400 students and 375 clinicians performing pelvic examinations using the simulator. The data include forces as a function of time recorded from sensors distributed in the simulator. In addition, background information on all of the users was also recorded. These records include a database of outcome measures, the user's clinical assessment scores, and independent skilled observer ratings of the users' techniques while performing examinations or procedures on the simulators.

### Data Analysis

The methodology for analyzing the data includes a multi-step processes of data reduction starting from multi-dimensional raw data and ending with a single objective performance score. The methodology is linked directly to the physics of the medium being treated. Data processing provides insights into the process being analyzed as opposed to a black box approach where only the inputs and outputs are well defined and the modal internal architecture is arbitrarily selected and unlinked to the physical world.

### Multi-Dimensional Raw Data

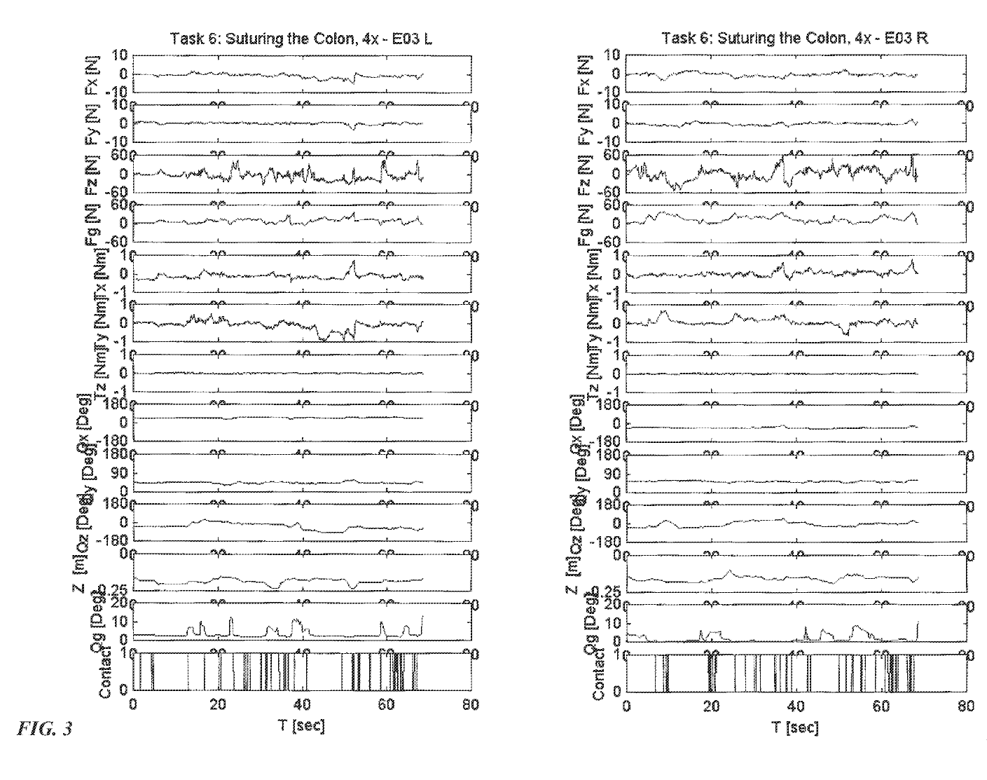

Multi-dimensional data was collected as a function of time for each modality under study. Time charts of the typical plots are depicted in FIG. 3. The exemplary data of FIG. 3 was acquired from the left and the right endoscopic tools of a surgical robot system during suturing of the colon by an expert surgeon in an MIS setup. Forces torques angles and contact information are plotted as a function of time.

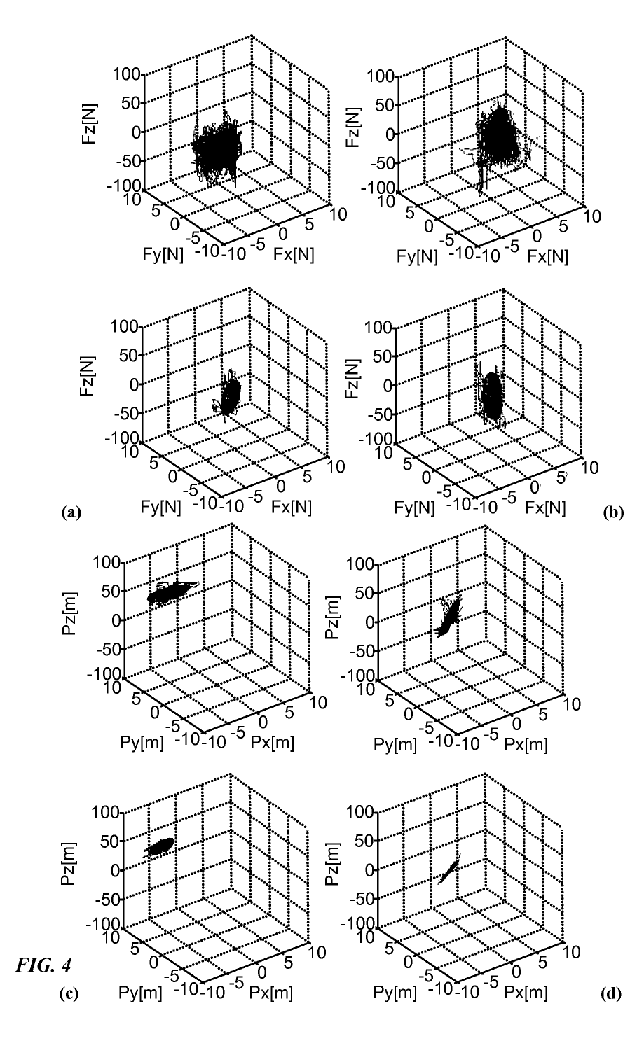

The vector representation of the data allows spatial graphical representation rather than time charts. Vector representation of exemplary data is shown in FIG. 4. The forces and torques (F/T) vectors are depicted as arrows with origins located at the port, and the lengths and orientations changing as a function of time based on the F/T applied by the surgeon's hand on the tool while interacting with the tissues, needle and suture. In a similar fashion, the traces of the tool tips with respect to the ports can be plotted as their positions changed during the surgical procedure using a spatial graphical form. Typical raw data of F/T and tool tip position traces were plotted using three dimensional graphs for the left and right endoscopic tools as measured by the surgical robot while performing the MIS intracorporeal knot tie by junior trainee (denoted as model R1 and shown in FIGS. 4A and 4C) and expert surgeon (denoted as model E and shown in FIGS. 4B and 4D). Forces are shown in FIGS. 4A and 4B and tool tip position is shown in FIGS. 4D and 4C. The ellipsoids contain 95% of the data points.

The complexity of the surgical task and the multi-dimensional data can be noted in the raw data. This complexity can be resolved, in part, by decomposing the surgical task into primary elements, thus enabling insights into the clinical procedure as a process.

### Vector Quantization

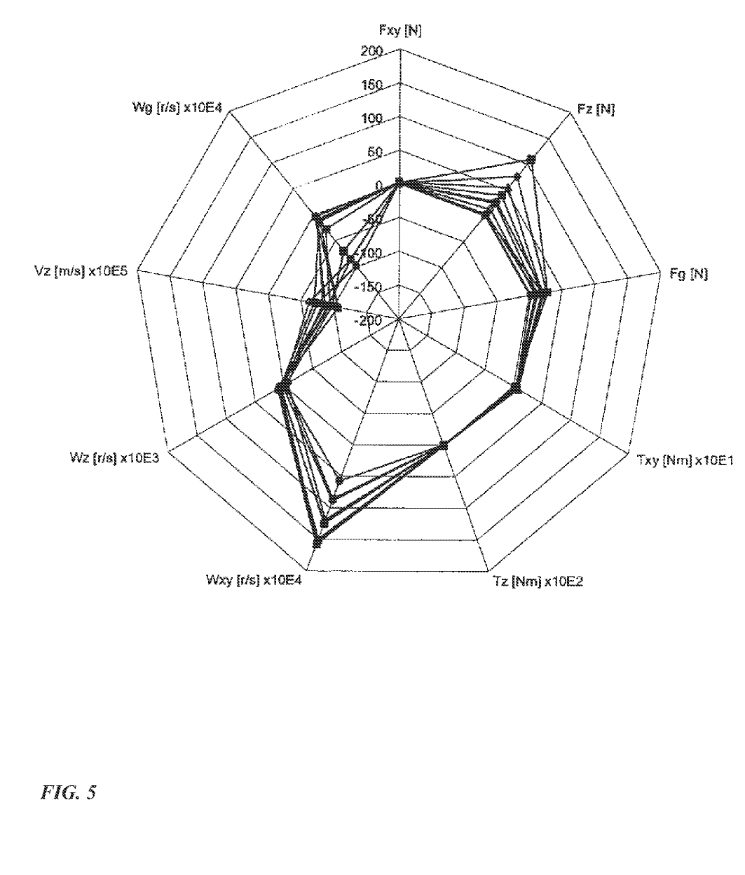

Data quantization is used to reduce the dimensions of the data. The data can be envisioned as a non-homogeneous discrete cloud encompassing the acquired data points, as illustrated in FIG. 5. As part of the iterative data quantization process, the vector quantization algorithm (e.g. K-means) searches for high-density regions in the non-homogeneous discrete cloud and assigns a cluster center to each one of the regions identified in the cloud. The number of clusters is bounded by the number of data points in the database (maximal value) and 1 (minimal value). In the extreme case where the number of clusters is equal to one, the cluster center vector represents the mean of that data. There are several techniques to define the optimal number of cluster centers in order to minimize the information that is lost due to data reduction associated with this process. Using the human language as an analogy, each data point associated with a specific cluster center represents a variant of a standard pronunciation defined by the cluster center.

Each cluster center can be defined by a discrete symbol (e.g. S̅₁, S̅₂, . . . , S̅_{K} etc.) forming a codebook. The database is then encoded into this codebook. Each point in the database is associated with only one cluster center in the codebook in which the distance between the selected cluster center and the data point is minimal. After encoding, the database contains a list of symbols as a function of time. The encoding process generates a substantial reduction in the dimensionality of the database. Encoding also reduces the data from a multi-dimensional space (e.g. 12 dimensional space in the case of the MIS database) to a single dimensional space of symbols (150 symbols in the case of the MIS database) representing the closest cluster centers as a function of time.

In one example, the number of states of a Markov model is selected based on user-selected criteria. For example, a 30-state Markov model can be used to represent two tools working collaboratively or a 3-state or 15-state hidden Markov model can be used to represent a single tool.

Each one of the 15 states was associated with a unique set of forces, torques, angular and linear velocities, as indicated in the table of FIG. 2. At various times, the tool might be in a specific state while infinite combinations of force, torque angular and linear velocities may be used. Data reduction is achieved by using a clustering analysis in a search for a discrete number of high concentration cluster centers in the database for each one of the 15 states. The continuous 13-dimensional vectors are transformed into one-dimensional vectors of 150 symbols (10 symbols for each state that was determined by the error distortion criterion).

Data reduction can be performed in three phases. During the first phase a subset of the database is created by appending the 13-dimensional vectors associated with each state measured by the left and the right tools and performed by all subjects. The 13-dimensional subset of the database ωₓ, ω_{y}, ω_{z}, ω_{g}, V_{z}, Fₓ, F_{y}, F_{z}, Tₓ, T_{y}, T_{z}, F_{g}, U) was transformed into a 9-dimensional vector x̅ᵢ=[ω_{xy}, ω_{z}, ω_{g}, V_{z}, F_{xy}, F_{z}, T_{xy}, T_{z}, F_{g}] by calculating the magnitude of the angular velocity, the forces and the torques in the X-Y plane (ω_{xy}=\sqrt{ωₓ²+ω_{y}²},F_{xy}=\sqrt{Fₓ²+F_{y}²},T_{xy}=\sqrt{Tₓ²+T_{y}²}). This process cancels out differences between surgeons due to variations in position relative to the animal and allowed the use of the same clusters for the left and the right tools. Note the tenth dimension U was omitted. This variable is used to differentiate the Idle state (State 1) in which the tool tip is not in contact with the tissue or other elements in the scene out of all the other states (states 2-15).

The subscripts x, y and z are used to associate the angular and linear velocities (ω, V), the forces (F), and torques (T) with the stationary coordinate system and an origin located at the surgical port. The combined axes x-y, x-z and y-z define planes parallel to the coronal, sagittal, and transverse planes respectively. The Z-axis is pointing toward the anterior side of the abdominal wall. The subscript g is used to associate the angular velocities (ω) and the forces (F) with the tool's grasping handle. The binary variable U indicates whether the tool is in contact with the tissue or any other element in the surgical scene.

In the second phase, a K-means vector quantization algorithm is used to identify 10 cluster centers associated with each state.

Mathematically the process is defined as follows: Given M patterns, X̅₁, X̅₂, . . . X̅_{M}, contained in the pattern space S̅, the process of clustering can be formally stated as seeking the regions S̅₁, S̅₂, . . . S̅_{K}, such that every data vector X̅ᵢ (i=1, 2, . . . , M) falls into one of these regions and no X̅ᵢ, is associated with two regions, i.e.

S̅₁U S̅₂U S̅₃ . . . U S̅_{K}= S̅ (a)

S̅₁I S̅ⱼ=0∀i≠j (b)(1)

The K-means algorithm is based on minimization of the sum of squared distances from all points in a cluster domain to the cluster center,

$$min {\sum }_{X ∈ {S}_{j} ( k )}^{ } {( \overline{X} - {\overline{Z}}_{j} )}^{2} \qquad ( 2 )$$

where sⱼ(k) was the cluster domain for cluster center Z̅ⱼ at the k^{th} iteration, and X̅ was a point in the cluster domain.

The cluster regions S̅ᵢ represented by the cluster centers Z̅ⱼ, defined typical signatures or codeword associated with a specific state (e.g. PS, PL, GR etc.). The number of clusters identified in each type of state is based upon the squared error distortion criterion (Equation 3). As the number of clusters increased, the distortion decreased exponentially. Following this behavior, the number of clusters is increased until the squared error distortion gradient, as a function of k, decreased below a threshold of 1% that results in at least 10 cluster centers for 14 out of the 15 states. Selecting the most frequent 10 clusters for each state guarantees that the squared error distortion gradient is 1% or smaller.

$$\begin{matrix}d ( \overline{X} , \overline{Z} ) = {‖ \overline{X} - {\overline{Z}}_{j} ‖}^{2} \\ = {\sum }_{i = 1}^{k} {( \overline{X} - {\overline{Z}}_{i} )}^{2}\end{matrix} \qquad ( 3 )$$

In a third phase, the 10 cluster centers Z̅ⱼ for each state forming a codebook of 150 discrete symbols were used to encode the entire database of the actual surgical tasks converting the continuous multi-dimensional data into a one-dimensional vector of finite symbols. This step of the data analysis facilitated the use of the discrete version of the Markov model.

FIG. 5 illustrates 10 cluster centers associated with a particular tool/tissue interaction (grasping-pushing-sweeping) in MIS as part of a codebook including 150 cluster centers representing a database of 5.5 millions data points. In grasping-pushing-sweeping, which is a superposition of three actions, the surgeon grasps a tissue or an object which is identified by the positive grasping force (F_{g}) acting on the tool's jaws and the negative angular velocity of the handle (ω_{g}) indicating that the handle is being closed. The grasped tissue or object is pushed into the port indicated by positive value of the force (F_{z}) acting along the long shaft of the tool and negative linear velocity (V_{z}) representing the fact that the tool is moved into the port. Simultaneously, sweeping the tissue to the side manifested by the force and the torque in the XY plane (F_{xy}, T_{xy}) that are generated due to the deflection of the abdominal wall, the lateral force applied on the tool by the tissue or object being swept along with the lateral angular velocity (ω_{xy}) indicating the rotation of the tool around the pivot point inside the port.

Ten signatures of forces, torques, linear and angular velocities are associated with the 15 types of states (tool/tissue or tool/object interaction) defined by the table illustrated in FIG. 2. Each one of the 10 polar lines represent one cluster. The clusters were normalized to a range of [−1, 1] using the following min/max values: ω_{xy}=0.593[r/s], ω_{z}=2.310[r/s], Vᵣ=0.059[m/s], ω_{g}=0.532[r/s], F_{xy}=5.069[N], F_{z}=152.536[N], F_{g}=33.669[N], T_{xy}=9.792[Nm], T_{z}=0.017[Nm].

In the graph of FIG. 5, each of the 10 polar lines represents one cluster. Each of the 15 other states or tool tissue/interactions defined in FIG. 2 is associated with 10 different and unique signatures defining a codebook with 150 symbols that can represent 5.5 million data points.

Both static, quasi-static and dynamic tool/tissue or tool/object interactions are represented by the various cluster centers. Even in static conditions, the forces and torques provide a unique and un-ambivalent signature that can be associated with each one of the 15 states.

### Markov Model

In one example, data analysis included developing a model that represents the process of performing MIS and methodology for objectively evaluating surgical skill. A Markov model provides a statistical method to summarize a relatively complex task such as a step or a task of a MIS procedure. In one example, skill level was incorporated into the Markov model by developing different models based on data acquired for different levels of expertise ranging from a first year resident to an expert surgeon.

A model is generated to represent the clinical procedure for analyzing the data. The model includes multiple interconnected states where each state represents an interaction between the physician using a tool or between the physician's hands and the tissues. After the physician is engaged in a specific interaction with the tissue, different forces, and torques (along with the tool kinematics) are generated through the interaction. The action/reaction information transmitted between the tool or the hand and the tissue is referred to as an observation and can be measured by an array of sensors incorporated into the various modalities previously noted.

The medical procedure can be described as a dynamic process in which the physician is moving between states while interacting with the tissue. During the physician's interaction with the tissue in each state, different types of information is exchanged between the tools (or the hand) and the tissue by utilizing the various observations typical to a specific state. After the physician is engaged with the tissue, the physician may remain in this state for a period of time and then perform a transition and engage with the tissue (again utilizing a different state), while using its associated observations.

This process can be modeled by a finite state machine or in a generalized form as a Markov model. The statistical nature of the model arises from the fact that each transition between two states or utilization of an observation in a state is associated with a probability. There is a particular probability that the physician will use certain transitions between the states that facilitates a specific observation while interacting in the tissue in a certain state. The model, as a whole, along with its states and observations, represents the clinical procedure. Moreover a specific navigation pattern between the model states and utilizing specific observations is associated with a particular skill. Physicians with a similar skill level are more likely to navigate through similar states of the model and leave the same trace. However, differences between the various skills level are related to different traces in the model. Each trace can be quantified by accumulating the probabilities associated with each transition. These accumulating probabilities define an objective score which can be used to differentiate between various skill levels.

The Markov model has a generic architecture (including the prime elements) such as states and observation. A specific model architecture defined for a particular medical procedure is based on an expert knowledge. Using expert knowledge, the various states and their interconnection are defined, and form a step in the model development. Each procedure has a unique model architecture and the generic methodology for assessing skill is independent of a specific procedure. The following sections will use MIS as an example of the methodology, thus demonstrating how the Markov model is translated into practice.

Analyzing the degrees of freedom (DOF) of a tool in MIS reveals that, due to the introduction of the port through which the surgeon inserts tools into the body cavity, two DOF of the tool are restricted. The six DOF of a typical open surgical tool is reduced to four DOF in a minimally invasive setup. These four DOF include rotation along the three orthogonal axes (x, y and z) and translation along the long axis of the tool's shaft (z). A fifth DOF is defined as the tool-tip jaws angle, which is mechanically linked to the tool's handle such as, when a grasper or a scissor is used. Additional one or two degrees of freedom can be obtained by adding a wrist joint to the MIS tool. The wrist joint enhances the dexterity of the tool within the body cavity.

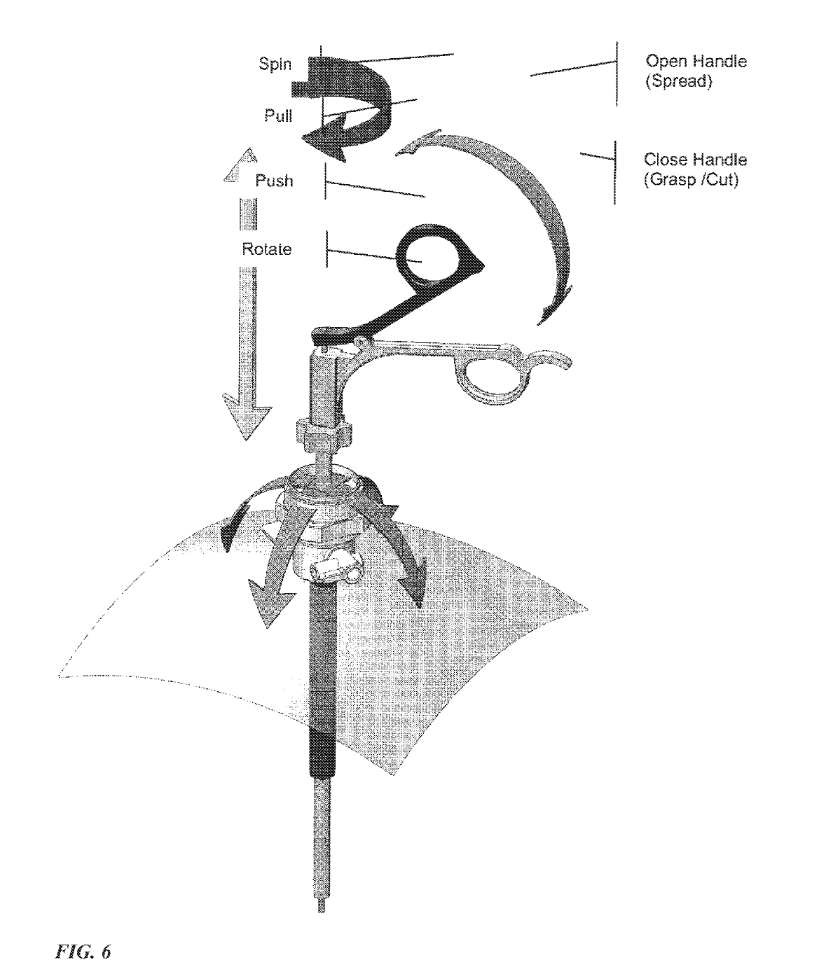

FIG. 6 illustrates five degrees of freedom in the context of a typical MIS endoscopic tool. Note that two DOF were separated into two distinct actions (Open/Close handle and Pull/Push), and the other two are combined into one action (Rotate) for representing the tool tip tissue interactions (omitted in the illustration). The terminology associated with the various DOF corresponds with the model state definitions noted in FIG. 2.

Surgeons, while performing MIS procedures, utilize various combinations of the DOF while manipulating the tool during the interaction with the tissues or other items in the surgical scene (such as a needle, a suture or a staple) in order to achieve the desired outcome. In one example, quantitative analysis of the position and orientation of the tool during surgical procedures revealed 15 different combinations of the five DOF for a tool while interacting with the tissues and other objects. These 15 DOF combinations will be further referred to, and modeled as states (see FIG. 2). The 15 states can be grouped into three types, based on the number of movements or DOF utilized simultaneously. The first type are fundamental maneuvers. The ‘idle’ state was defined as moving the tool in space (body cavity) without touching any internal organ, tissue or other item in the scene. The forces and torques developed in this state represent the interaction with the port and the abdominal wall, in addition to the gravitational and inertial forces. In the ‘grasping’ and ‘spreading’ states, compression and tension were applied on the tissue through the tool tip by closing and opening the grasper's handle, respectively. In the ‘pushing’ state, the tissue was compressed by moving the tool along the Z-axis. ‘Sweeping’ consisted of placing the tool in one position while rotating it around the X- and/or Y-axes or in any combination of these two axes (port frame). State 15 was observed in tasks involving suturing when the surgeon grasps the needle and rotating it around the shaft's long axis to insert it into the tissue. Such a rotation was not observed whenever tissue interaction was involved. With the exception of state 15, the rest of the tool/tissue interactions in Types II and III were combinations of the fundamental ones defined as Type I.

The modeling approach underlying the methodology for decomposing and statistically representing a surgical task is based on a fully connected, symmetric finite-states (30 states) Markov model where the left and the right tools are represented by 15 states each as illustrated in FIG. 5. Each one of the 15 states corresponds to a fundamental tool/tissue or tool/object interaction based on tool kinematics and is associated with unique F/T and velocity signatures defined as observations and measured at the hand/tool interface and then translated to the port coordinate system of FIG. 2. In view of this model, a minimally invasive surgical task can be described as a series of finite states. In each state, the surgeon is applying a specific force/torque/velocity signature, out of 10 signatures that are associated with that state, on the tissue or on another item in the surgical scene by using the tool. The surgeon may stay within the same state for a specific time duration using different signatures associated with that state and then perform a transition to another state. The surgeon may utilize any of the 15 states by using the left and the right tools independently. The states representing the tool/tissue or tool/object interactions of the left and the right tools are mathematically and functionally linked.

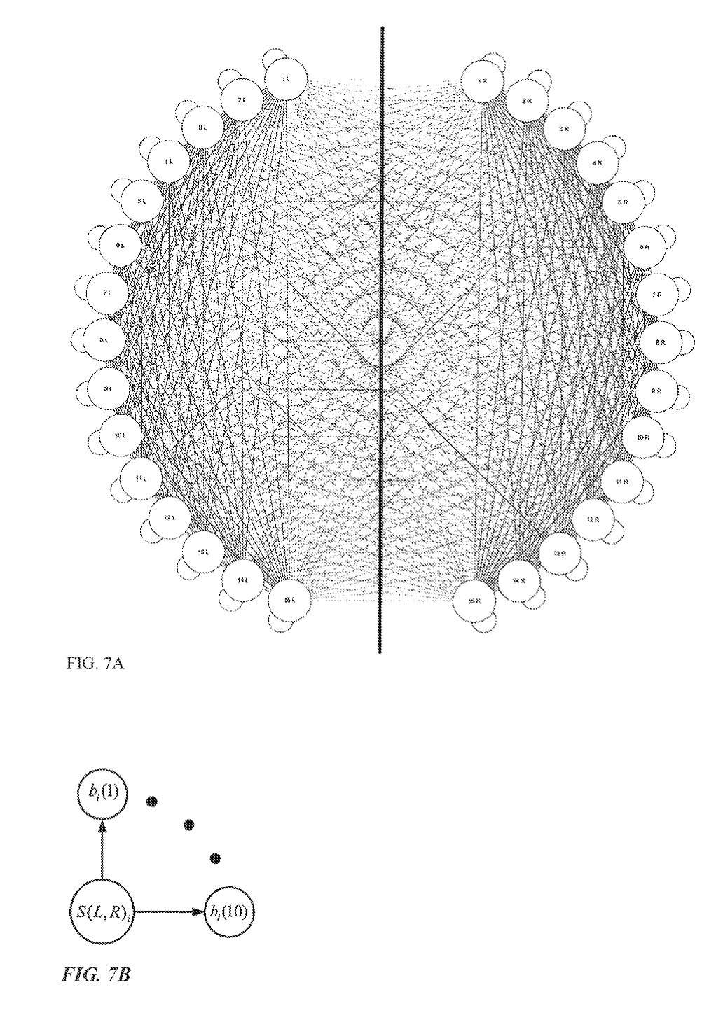

FIG. 7A illustrates a fully connected finite state diagram (FSD) for decomposing MIS. The tool/tissue and tool/object interactions of the left and the right endoscopic tools are represented by the 15 fully connected sub-models. Circles represent states whereas lines represent transitions between states. Each line that does not cross the center-line represents a probability value defined in the state transition probability distribution matrix A={aᵢⱼ}. Each line that crosses the center line represents a probability for a specific combination of the left and the right tools and is defined by the interstate transition probability distribution matrix or the cooperation matrix C={cₗᵣ}. Note that since the probability of performing a transition from state i to state j by each one of the tools is different from probability of performing a transition from state j to state i, these two probabilities could have been represented by two parallel lines connecting state i to state j and representing the two potential transitions. For purposes of simplifying the graphical representation of A={aᵢⱼ} only one line is plotted between state i to state j.

FIG. 7B illustrates that each state out of the 15 states of the left and the right tool b(L,R)ᵢ, is associated with the 10 force/torque/velocity signatures or discrete observations bᵢ(1) . . . bᵢ(10). Each line that connects the state with a specific observation represents a probability value defined in the observation symbol probability distribution matrix B={bⱼ(k)}. The sub-structure associated with each state (b) is omitted to simplify the diagram.

The Markov model is defined by the notation in Equation 4. Each Markov sub-model representing the left and the right tool is defined by λ_{L} and λ_{R} (Equation 4). The sub-model is defined by:

(i) The number of states—N whereas individual states are denoted as S={s₁, s₁, . . . s_{N}}, and the state at time t as qₜ;

(ii) The number of distinct (discrete) observation symbol—M whereas individual symbols are denoted as V={v₁, v₁, . . . v_{M}};

(iii) The state transition probability distribution matrix indicating the probability of the transition from state qₜ=sᵢ at time t to state qₜ₊₁=sⱼ at time t+1−A={aᵢⱼ}, where aᵢⱼ=P[qₜ₊₁=sⱼ|qₜ=sᵢ] 1≦i,j≦N;

Note that A={aᵢⱼ} is a non-symmetric matrix (aᵢⱼ≠aᵢⱼ) since the probability of performing a transition from state i to state j using each one of the tools is different from the probability of performing a transition from state j to state i.

(iv) The observation symbol probability distribution matrix indicating the probability of using the symbol vₖ, while staying at state sⱼ at time t−B={bⱼ(k)}, where for state j bⱼ(k)=P[vₖ at t|qₜ=sⱼ] 1≦j≦N, 1≦k≦M;

(v) The initial state distribution vector indicating the probability of starting the process with state sᵢ at time t=1−π where πᵢ=P[q₁=sᵢ] 1≦i≦N.

The two sub-models are linked to each other by the left-right interstate transition probability matrix or the cooperation matrix indicating the probability for staying in states sₗ with the left tool sᵣ with the right tool at time t−C={cₗᵣ}, where cₗᵣ=P[q_{tL}=sₗ∪q_{tR}=sᵣ]1≦l,r≦N.

Note that C={Cₗᵣ} is a non-symmetric matrix (Cₗᵣ≠cᵣₗ) since it representing the combination of using two states simultaneously by the left and the right tools.

The probability of observing the state transition Q={q₁, q₂, . . . q_{T}} and the associated observation sequence O={o₁, o₂, . . . oₜ}, given the two Markov sub-models (Equation 4) and interstate transition probability matrix, is defined by Equation 5

$$\begin{matrix} {λ}_{L} = ( {A}_{L} , {B}_{L} , {π}_{L} ) \\ {λ}_{R} = ( {A}_{R} , {B}_{R} , {π}_{R} ) \\ {a}_{ij} = \frac{n ( {q}_{t} = {s}_{j} \vert {q}_{t - 1} = {s}_{i} )}{n} \\ {b}_{jk} = \frac{m ( {v}_{k} \vert {q}_{t} = {s}_{j} )}{m ( {q}_{t} = {s}_{j} )} \\ {c}_{lr} = \frac{c [ {q}_{Lt} = {s}_{l} ⋃ {q}_{Rt} = {s}_{r} ]}{n} \\ \begin{matrix} {\sum }_{j = 1}^{N} {a}_{ij} = {\sum }_{k = 1}^{M} {b}_{jk} \\ = {\sum }_{l = 1 , r = 1}^{l = N , r = N} {c}_{lr} \\ = 1\end{matrix} & ( 4 ) \\ P ( Q , O \vert {λ}_{L} , {λ}_{R} , C ) = {π}_{{q}_{L}} {π}_{{q}_{R}} {\prod }_{t = 0}^{T} {a}_{{q}_{t} {q}_{t + 1} L} {b}_{{q}_{t} L} ( {o}_{t} ) {a}_{{q}_{t} {q}_{t + 1} R} {b}_{{q}_{t} R} ( {o}_{t} ) {c}_{{q}_{t} L} {q}_{tR} & ( 5 )\end{matrix}$$

Since probabilities, by definition, have numerical value in the range of 0 to 1, the probability calculated by Equation 5 converges exponentially to zero and therefore exceeds the precision range of a machine. Hence, by using logarithmic transformation, the resulting values of Equation 5 in the range of [0 1] are mapped by Equation 6 into [−∞0].

$$Log ( P ( Q , O \vert {λ}_{L} , {λ}_{R} , C ) ) = Log ( {π}_{qL} ) + Log ( {π}_{qR} ) + {\sum }_{t = 1}^{T} Log ( {a}_{{q}_{t} {q}_{t + 1} L} ) + Log ( {b}_{{q}_{t} L} ( {o}_{t} ) ) + Log ( {a}_{{q}_{t} {q}_{t + 1} R} ) + Log ( {b}_{{q}_{t} R} ( {o}_{t} ) ) + Log ( {c}_{{q}_{t} {Lq}_{t} R} ) \qquad ( 6 )$$

Due to the nature of the process associated with surgery in which the procedure, by definition, always starts in the idle state (state 1), the initial state distribution vector is defined as follows in Equation 7.

π_{1L}=π_{1R}=1

π_{iL}=π_{iR}=0 2≦i≦N. (7).

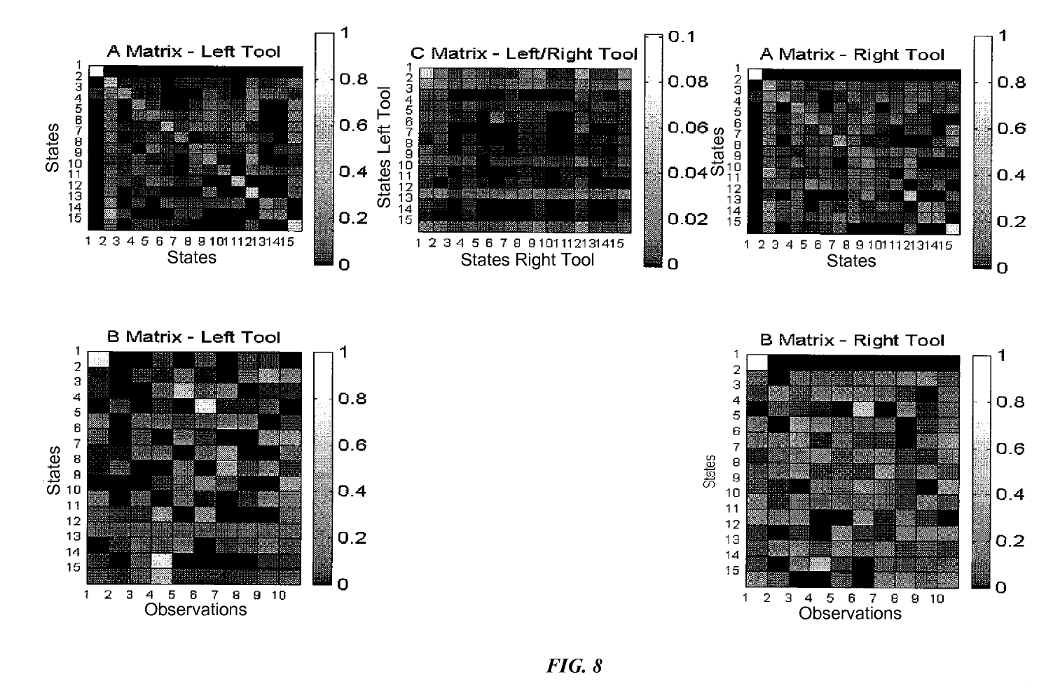

Given the encoded data, 30 Markov models, (one for each subject) are calculated defining the probabilities for performing certain tool transitions ([A] matrix), the probability of combining two states ([C] matrix), and the probability of using the various signatures in each state ([B] matrix). FIG. 8 illustrates an exemplary Markov model where the matrices [A], [B], [C], are represented as coded probabilistic maps.

An element in the [A] matrix is calculated as the ratio between the number of times a specific transition between state i to state j took place n(qₜ=sⱼ|qₜ₋₁,=sᵢ) and the total number of state transitions n which is also equal to one minus the number of data points. There are N numbers of potential transitions between two states and therefore the order of [A] is N×N. The sum of each line in the [A] matrix is equal to one. An element in the [B] matrix is calculated as the ratio between the number of times a specific observation vₖ was used while staying in state sⱼ, m(vₖ|qₜ=sᵢ) and the total number of visits of state j, m(qₜ=sⱼ) which is also equal to the number of times any observation was used while visiting that state. There are N number states and M number of potential transitions between two states and therefore the order of [A] is N×N. The sum of each line in the [B] matrix is equal to one. An element in the [C] matrix is calculated as the ratio between the number of times the left hand side model is in state sₗ as well as the right hand side of the model is in state sᵣ, c(q_{Lt}=sₗ∩q_{Rt}=sᵣ) and the total number of state combinations observed n which is also equal to the number of data points. The sum of all lines and columns of the [C] matrix is equal to one.

In models extracted as described above from the sample surgical data, the highest probability values in the [A] matrix appear along the diagonal. Accordingly, a transition associated with remaining at the same state is more likely to occur rather than a transition to any one of the other 15 potential states. In minimally invasive surgical suturing, for example, the default transition from any state is to the grasping state (state number 2) as indicated by the high probability values along the second column of the [A] matrix. The probability of using one out of the 150 cluster centers (illustrated in FIG. 5) is graphically represented by the [B] matrix. Each line of the [B] matrix is associated with one of the 10 states. The clusters were ranked according to the mechanical power. The left and the right tool used different distribution of the clusters. With the left tool, the most frequent clusters that were used are related to mid-range power and with the right tool, the cluster usage is more evenly distributed among the different power levels. The collaboration matrix [C] indicates that the most frequently used state with both the left and the right tools are idle (state 1), grasping (state 2), and grasping pulling and sweeping (state 12). In addition, grasping rotating (state 15) with the left tool was also frequently used. Once one of the tools utilizes one of these states, the probability of using any of the states by the other tool is equally distributed between the states which is indicated by the bright stripe in the graphical representation of the [C] matrix.

Each tool (left and right) can be only in one out of the 15 states. However, there are potentially 225 (15×15) different combinations in which the left tool is in state i and the right tool is in state j. For that reason the dimensions of the [C] matrix is 15×15.

The idle state (state 1) in which no tool/tissue interaction is performed was mainly used, in most of the surgical tasks (by both expert and novice surgeons), to move from one operative state to another. The expert surgeons used the idle state as a transition state while the novices spent a significant amount of time in this state planning the next tool/tissue or tool/object interaction. In the case of surgical suturing and knot tying, the grasping state (state 2) dominated the transition phases since the grasping state, in this case, maintains the scene in an operative state in which both the suture and the needle were held by the two surgical tools.

### Objective Skill Assessment

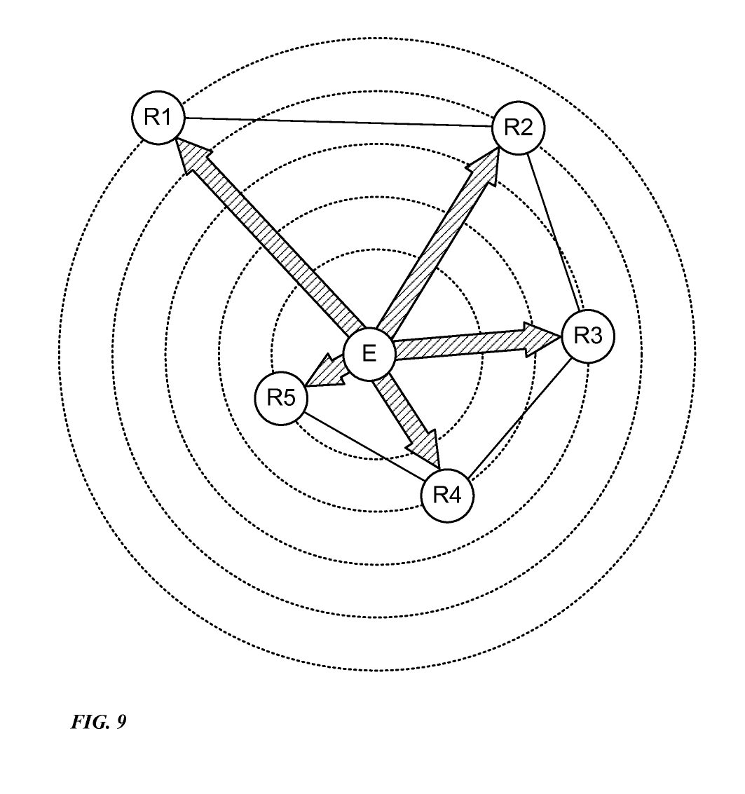

Once the Markov models are defined for specific subjects with specific skill levels, it becomes possible to calculate the statistical distance factors between them. The statistical distance factors are considered to be an objective criterion for evaluating skill level if for example, the statistical distance factor between a trainee (indicated by index R) and an expert (indicated by index E) is being calculated. FIG. 9 illustrates a schematic representation of the statistical distance between and expert (E) and residents (R1 . . . R5) as represented by the arrows. The statistical similarity is changing as a function of training time (moving clockwise about the expert) along as the subject's performance becomes similar to the experts' performance. The statistical distance indicates similarity as to the performance of the two subjects under study.

Given two Markov models λ_{Ei}=λ_{LEi},λ_{REi},C_{Ei}) (expert) and λ_{Ti}=(λ_{LTj},λ_{RTj},C_{Tj}) (trainee) the asymmetric statistical distances between them are defined as D₁(λ_{Tj},λ_{Ei}) and D₂ (λ_{Ei},λ_{Tj}). The natural expression of the symmetric statistical distance version D_{EiTi} is defined by Equation 8.

$${D}_{EiTj} = \frac{{D}_{1} ( {O}_{Ei} , {Q}_{Ei} , {O}_{Tj} , {λ}_{Ei} ) + {D}_{2} ( {O}_{Ei} , {Q}_{Ei} , {O}_{Tj} , {Q}_{Tj} , {λ}_{Tj} )}{2} = \frac{1}{2} ( \frac{log P ( {O}_{Tj} , {Q}_{Tj} \vert {λ}_{Ei} )}{log P ( {O}_{Ei} , {Q}_{Ei} \vert {λ}_{Ei} )} + \frac{log P ( {O}_{Tj} , {Q}_{Tj} \vert {λ}_{Tl} )}{log P ( {O}_{Ei} , {Q}_{Ei} \vert {λ}_{Tj} )} ) \qquad ( 8 )$$

Setting an expert level as the reference level of performance, the symmetric statistical distance of a model representing a given subject from a given expert (D_{EiTj}) is normalized with respect to the average distance between the models representing all the experts associated with the expert group ( D̅_{EE}) in Equation 9. The normalized distance ∥D_{EiTj}∥ represents how far (statistically) is the performance of a subject, given his or her model, from the performance of the average expert.

$$‖ {D}_{EiTj} ‖ = \frac{{D}_{EiTj}}{{\overline{D}}_{EE}} = \frac{{D}_{EiTj}}{\frac{1}{l} {\sum }_{u = 1 ; v = 1}^{u = 5 ; v = 5} {D}_{{E}_{u} {E}_{v}}} for u \neq v \qquad ( 9 )$$

For the purpose of calculating the normalized learning curve, the distances between all the subjects associated with the group of experts was first calculated D_{E}_{ᵤ}_{E}_{ᵥ}−(for five subjects in the expert group−u=v=1 . . . 5−=20) using Equation 8. The denominator of Equation 9 was then calculated.

Once the reference level of expertise was determined, the statistical distances between each one of the 25 subjects, grouped into five levels of training (R1, R2, R3, R4, R5), and each one of the experts was calculated (5 distances for each individual, 25 distances for each group of skill level and 125 distances for the entire data base) using Equation 8. The average statistical distance and its variance define the learning curve of a particular task.

### Complimentary Objective Indexes

In addition to the Markov models and the statistical similarity analysis, two other objective indexes of performance can be measured and calculated, including the task completion time and the overall length (L) of the path generated by the left and the right tool tips. Where D_{L}, D_{R}, are the distances between two consecutive tool tip positions P_{L}(t−1), P_{R}(t−1) and P_{L}(t),P_{R}(t) as a function of time of the left and the right tools respectively.

$$L = {\sum }_{t = 1}^{T} {D}_{L} ( {P}_{L} ( t - 1 ) , {P}_{L} ( t ) ) + {D}_{R} ( {P}_{R} ( t - 1 ) , {P}_{R} ( t ) ) \qquad ( 10 )$$

These complimentary performance indexes are available for the particular surgical robot database in which motion of the tool was acquired. Acquisition of tool motion in the other modalities is also contemplated.

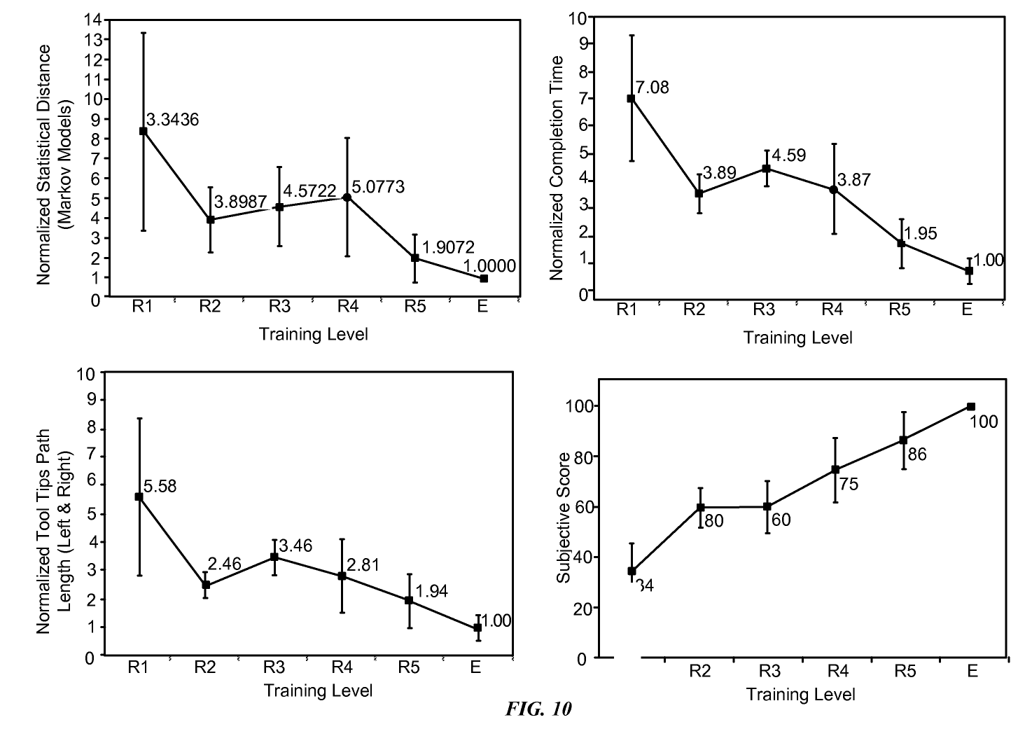

FIGS. 10A-C illustrate normalized Markov model-based statistical distance as a function of the training level, normalized completion time and normalized path length of the two tool tips respectively. The complementary subjective normalized scoring is depicted in FIG. 10D.

In particular, FIG. 10 illustrates objective and subjective assessment indexes of minimally invasive suturing learning cure. The objective performance indexes are based on: (a) Markov model normalized statistical distance, (b) normalized completion time, and (c) normalized path length of the two tool tips. In the example illustrated, the average task completion time of the expert group is 98 seconds and the total path length of the two tools is 3.832 m. The subjective performance index is based on subjective scoring of the tasks' videos and normalizing the score with respect to experts' performance (d).

The data illustrates that substantial suturing skills are acquired during the first year of the residency training. The learning curves do not indicate significant improvement during the second and the third years of training. The rapid improvement of the first year is followed by lower gradient of the learning curve as the trainees progress toward the expert level. The Markov model-based statistical distance along with the completion time criteria indicate another gradient in the learning curve that occurs during the fourth year of the residency training followed by slow conversion to expert performance. Similar trends in the learning curve are also demonstrated by the subjective assessment. One particular subject in the R2 group outperformed his peers in his own group and some subjects in a more advanced groups (R3, R4) which slightly altered the overall trend of the learning curves as defined by the different criteria.

### Exemplary Method

An exemplary method includes the following steps: (a) acquire raw performance data; (b) use the K-means algorithm (software) to identify clusters in the database; (c) encode the entire databases using the clusters identified in (b); (d) define a Markov model for each subject performing a specific task; (e) calculate the statistical distances between the Markov models representing subjects with various skill levels and correlate these measurements with the known skill levels while defining the learning curves; and (f) to optionally validate the method of steps (a-e), perform the complimentary analysis (time, path length subjective assessment) and correlate the results with the Markov analysis (objective assessment).

### Application

A clinical procedure, regardless of the performance modality, entails synthesis between visual and kinesthetic information. Analyzing the procedure in terms of these two sources of information facilitates development of objective criteria for training physicians and evaluating the performance in different modalities including real procedures, master/slave robotic systems or virtual reality or physical simulators.

The Markov model and the vector quantization described herein is suitable for multi-modal sources of information, including low level data (such as tool kinematics and dynamics defining the model observations) and high level methodological processes (such as tool/tissue interactions formulating the model's state). The Markov model provides a mathematical representation of the process associated with manipulative tasks including complex medical procedures such as surgery. In one example, the present subject matter provides a quantitative and objective measure of surgical performance.

Exemplary outcomes of analysis of minimally invasive surgical procedures using the present subject matter revealed differences between surgeons at different skill levels including, (i) the types of tool/tissue/object interactions being used; (ii) the transitions between tool/tissue/object interactions being applied by each hand; (iii) time spent while performing each tool/tissue/object interaction; (iv) the overall completion time; (v) the various F/T/velocity magnitudes being applied by the subjects through the endoscopic tools; and (vi) two-handed collaboration. In addition, the F/T associated with each state revealed that the F/T magnitudes are relatively task-dependent with relatively high F/T magnitudes applied by novices compared to experts during tissue manipulation, and vice versa during tissue dissection. High efficiency of surgical performance was demonstrated by the expert surgeons and expressed by shorter tool tip displacements, shorter periods of time spent in the ‘idle’ state and sufficient application of F/T on the tissue to safely accomplish the task.

In various examples, the present subject matter facilitates development of objective criteria for decomposing a medical procedure and analysis using models. In one example, objective measures of skill and competency enables training and evaluating performance. In real-time, the present subject matter provides feedback to the trainee or as an artificial intelligent background layer which may increase performance efficiency in medicine and improve patient safety and outcome.

### Indexes of Performance

Following two steps of data reduction, data that were collected by the surgical robot and were used to develop models representing MIS as a process. In data reduction, there is a compromise between decreasing the input dimensionality while retaining sufficient information to characterize and model the process under study. Utilizing the VQ algorithm the 13 dimensional stream of acquired data were quantized into 150 symbols with nine dimensions each.

The data quantization included identification of the cluster centers and encoding the database based on the identified cluster centers. Every data point meeting two criteria is then associated with one of the 150 identified cluster centers. The first criterion is to have the minimal geometrical distance to one of the cluster centers. Once the data point was associated with a specific cluster center it is, by definition, associated with a specific state out the 15 defined. Based on expert knowledge of surgery, the table in FIG. 2 defines the 15 states and unique sets of individual vector components. The second criterion is that, given the candidate state and the data vector, the direction of each component in the vector must match the one defined by the table for the selected state. It was indicated during the data processing that these two criteria were typically met suggesting that the data quantization process is very robust in it nature. Following the encoding process a 2-dimensional input (one dimension for each tool) was utilized to form a 30 state fully connected Markov model. The coded data with their close association to the measured data, as well as the Markov model using these codes as its observations distributed among its states, retain sufficient multi-model information in a compact mathematical formulation for modeling the process of surgery at different levels.

MIS is recognized both qualitatively and quantitatively as a multidimensional process. As such, studying one parameter (e.g. completion time, tool-tip paths, or force/torque magnitudes) reveals only one aspect of the process. A model that describes MIS as a process can facilitate study of the internal process and provide information. At the high level, a tremendous amount of information is encapsulated into a single objective indicator of surgical skill level and expressed as the statistical distance between the surgical performance of a particular subject under study from a surgical performance of an expert. As part of an alternative approach a combined score could be calculated by studying each parameter individually (e.g. force, torque, velocity, tool path, completion time etc.), assigning a weight to each one of these parameters, which is a subjective process by itself, and combining them into a single score. The assumption underlying this approach is that a collection of aspects associated with surgery may be used to assess the overall process. However this alternative approach ignores the internal process that is more likely to be revealed by a model such as the Markov model. In addition, as opposed to analyzing individual parameters, studying the low levels of the model provides profound insight into the process of MIS in a way that allows one to offer constructive feedback for a trainee regarding performance aspects like the appropriate application of F/T, economy of motion, and two handed manipulation.

The application of F/T on the tissue has an impact on the surgical performance efficiency and outcome of surgery. Some results indicate that the F/T magnitudes are task dependent. Experts applied high F/T magnitudes on the tissues during tissue dissection as opposed to low F/T magnitudes applied on the tissues by trainees that were trying to avoid irreversible damage. An inverse relationship regarding the F/T magnitudes was observed during tissue manipulation in which high F/T magnitudes applied on the tissue by trainees exposed them to acute damage. These differences were observed in particular states (e.g. those states including grasping for tissue manipulation and states involving spreading for tissue dissection). Due to the inherent variance in the data, even multidimensional ANOVA failed to identify this phenomena once the F/T magnitudes are removed from the context of the multi-state model. Given the nature of the surgical task, the Markov model [B] Matrix, encompassing information regarding the frequency in which the F/T magnitudes were applied, may be used to assess whether the appropriate magnitudes F/T were applied for each particular state. Tissue damage is correlated with surgical outcome and linked to the magnitudes and the directions in which F/T were applied on the tissues. As such, tissue damage boundaries may be incorporated into the [B] matrix for each particular state. Given the surgical task, this additional information may refine the constructive feedback to the trainee and the objective assessment of the performance.

The economy of motion and the two hand collaboration may be further assessed by retrieving the information encapsulated into the [A], and [C] matrices. The amount of information incorporated into these two data structures exceeds the information provided by a single indicator (such as tool-tip path length or completion time) for the purpose of formulating constructive feedback to the trainee. Given a surgical task, utilizing the appropriate sets of states and state transitions are skill dependent. This information is encompassed in the [A] matrix indicating the states that were in use and the state transitions that were performed. Moreover, the ability to refine the time domain analysis using the multi-state Markov model indicated, as was observed in previous studies, that the ‘idle’ state is utilized as a transition state by expert surgeons whereas a significant amount of time is spent in that state by trainees.

Coordinated movements of the two tools is yet another indication of high skill leveling MIS. At a lower skill level the dominant hand is more active than the non-dominant hand as opposed to a high skill level in which the two tools are utilized equally. The collaboration [C] matrix encapsulates this information and quantifies the level of collaboration between the two tools.

The Markov model provides insight into the process of performing MIS. This information can be translated into a constructive feedback to the trainee as indicated by the three model matrices [A], [B] and [C]. Moreover, the capability of running the model in real-time and its inherent memory allows a senior surgeon supervising the surgery or an artificially intelligent expert system incorporated into a surgical robot or a simulator to provide immediate constructive feedback during the process as previously described.

Although the notations and the model architecture of the Markov model and the hidden Markov model approach are similar, there are several differences between them. The Markov model can be perceived as a white box model in which each state has a physical meaning describing a particular interaction between the tools and tissue or other objects in the surgical scene (such as sutures and needles). The hidden Markov model can be perceived as a black box model in which the states are abstract and are not related to a specific physical interaction. In the white box model, each state has a unique set of observations that characterize only the specific state. By definition, once the discrete observation is matched with a vector quantization code-word the state is also defined. States in the hidden Markov model share the same observations, however different observation distributions differentiate between them.

### Additional Examples

Other sensors can be used to generate data for the present subject matter including, for example, sensors configured to measure position, orientation, force, torque, pressure, physiological variables and contact. In addition, other sensors, including a velocity sensor, an acceleration sensor, a pressure sensor, a visual display of a scene being analyzed, a clock, and a temperature sensor can also be used to generate data for the present subject matter.

In one example, a hybrid model is generated which represents the topology between a Markov model and a hidden Markov model. The hybrid model adds another layer of complexity to the Markov model by introducing the observation elements for each state. The hybrid model provides insight into the process by linking the states to physical and meaningful interactions. The hybrid model includes the collaboration matrix [C] in addition to the Markov model notation. The collaboration matrix [C] is not normally present in either the Markov model or the hidden Markov model. The collaboration matrix [C] links the models representing the left and right hand tools since surgery is a two-handed task.

In one example, the Markov model provides physical meaning to the process being modeled. In one example, the hidden Markov model provides a compact model topology and does not rely on expert knowledge incorporated into the model.

In one example, a method of the present subject matter includes defining the scope of the model and the fundamental elements, the state and the observation. For example, in the case of minimally invasive surgery, the surgical task is modeled by a fully connected model topology were each tool/tissue/object interaction is modeled as a state. In one example, each phenomenon is represented by a model with abstract states wherein each tool/object interaction is modeled by an entire model using more generalized definitions for these interactions e.g. place position, insert remove. In one example, additional models are used with a predetermined overall structure that represents the overall process.

In one example, the scope of the model is limited to objectively assess technical factors of surgical ability. Cognitive factors can be assessed by the model where a specific action is taken as a result of a decision making process.

Decomposing MIS and analyzing it using a Markov model is one approach for developing objective criteria for surgical performance.

In one example, the present subject matter, when used in real-time during the course of learning as feedback to the trainee surgeons or as an artificial intelligent background layer, may increase performance efficiency in MIS and improve patient safety and outcome.

One example of the present subject matter utilizes a plurality of models and a performance of a specimen is correlated to a particular model based on a generated distance that describes the probability that the specimen matches a particular one of the plurality of models.

The present subject matter can be applied to other types of human machine interfaces, including, for example, flight simulators and vehicle simulators and other multi-state non-medical devices and simulators.

In one example, an intelligent layer or expert system is configured to interject a message or interrupt a process performed by a robotic device. For example, an imprudent manipulation by a low skilled surgeon will trigger delivery of a message, either visually, audibly or tactile. In one example, the robotic device will prevent an imprudent manipulation or provide cues to suggest adoption of an alternate manipulation.

In one example, the models are adapted or trained against a data set. For example, a first year resident performing a minimally invasive surgical procedure will generate a particular set of performance data. In one example, a Baum-Welch algorithm is executed by a set of computer implemented instructions. A Baum-Welch algorithm is used to train the models for each skill level based on data from the training groups of known skill levels. In other words, the Baum-Welch algorithm facilitates the determination that the hidden Markov model can generate data matching the particular specimen performance. The Baum-Welch algorithm is but one example of a class of algorithms known as forward-backward algorithms, machine learning algorithms or pattern recognition algorithms and other algorithms are also contemplated for use with the present subject matter. In one example, a forward-backward algorithm is used to determine the probability that the specimen performance correlates to a particular Markov model.

In one example, the surgical robot is equipped with 26 sensors and at a sampling rate of 100 readings per second, 2,600 data points are generated per second.

Execution of the Baum-Welch algorithm facilitates adaptation or modification of the data to represent a particular subject performance. In one example, the Baum-Welch algorithm is executed for each particular skill level in order to train the model. In one example, specimen data is used in the forward-backward algorithm and applied to the data corresponding to each of the six models generated and the present subject matter selects the one model with the highest probability. In one example, a correlation function is executed to determine a performance grade for a particular specimen.

In one example, a “distance” is calculated between each mode and the specimen data set. The shortest distance correlates to the highest probability for a match.

In one example, a recurrent neural networks (ARMA, autoregressive moving average) is calculated to correlate specimen performance to a particular model data set.

In various examples, measurements of the tool path length (a measure of the movement of a tool tip), time, force applied or other parameter is used to judge performance. Other parameters include torque, position, displacement, electrical contact measurement (resistance) and temperature. Such parameters can be used in the analysis of surgical tasks such as suturing, cutting, cauterizing and ablating.

In one example, a hidden Markov model is applied to physical signals generated by a performance of a manipulative task conducted by a specimen. The internal parameters are adjusted to improve stability of the signal generated. For example, a window is established around a particular signal to a limit the amount of variable changes. By establishing a window or boundaries, the asymptotic change of a value is bracketed and convergence is accelerated. In one example, a trial and error approach is performed in establishing the boundaries for a particular signal value.

The present subject matter can be operated in real-time and provide feedback (any of visual, aural, tactile) regarding performance during the manipulative task.

The methodology is independent of the modality used and can be incorporated into an example of the present subject matter including any of an instrumented surgical tool, a simulator, and a robotic system. In addition, the present subject matter can include an instrumented tool configured to provide performance data where the tool is a non-surgical device.

In one example, the present subject matter executes an algorithm that can be described as a black box model of skill. The black box model generates generalized findings such as probabilities, fuzzy logic membership functions, or similar abstract numbers. In one example, the algorithm generates generalized findings of skill using a model based on fuzzy logic.

### Spherical Motion Mechanism

The present subject matter further includes a spherical motion mechanism suitable for a variety of applications, including for example, manipulating a minimally invasive surgery (MIS) instrument. The mechanism includes a first revolute joint connected to a link with a less than right angle between the link and the axis of rotation for the first revolute joint. At the other end of the first link is a second revolute joint with a less than right angle between the first link and the axis of rotation for the second revolute joint and a less than right angle between the axis of rotation and the second link. Additional links can be added at the end of the first two links.

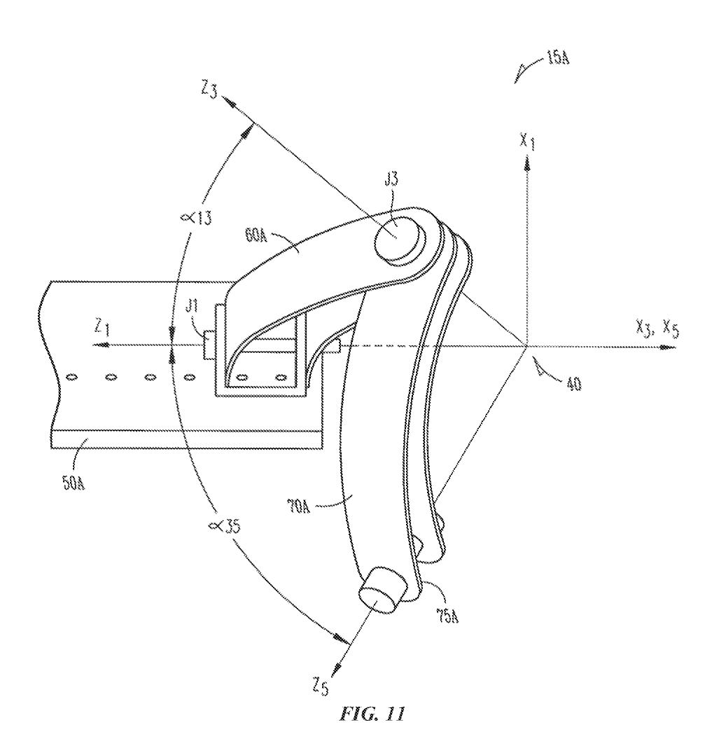

In one example, a device includes two links configured as described and illustrated in FIG. 11. The axes of rotation of the revolute joints converge, or alternatively intersect, at a point remote from the device. This intersection constrains the end link or effector to move about the surface of a sphere where the center of the sphere lies at the intersection of the axes of rotation.

In one example, the device includes a single series of two links however multiple links can be used wherein additional links are connected at the last link to form a parallel version.

A load can be placed at the end link. In various examples, the load includes a manipulator, an optical device, an audio device, a probe, an electrical device or other such device. The device focuses at the center of the sphere.

In one example, the load includes a tool or surgical instrument having a longitudinal tool axis. The instrument can be aligned such that the longitudinal axis passes through the center of the sphere. Rotation of the instrument about the longitudinal tool axis then passes through the center as well as any prismatic motion of the instrument. The center or origin of the sphere can be placed coincident with the MIS trocar.

In one example, the device is used as an imager. The center of the sphere can be located inside a patient (since the center of the sphere is separate and remote from the device) and an image of a focused area inside the patient can be created. The focused area can be at or about the center of the sphere.

In addition to surgical and medical applications, the device can be configured for manipulating and assembling small scale components.

In one example, subject matter also includes a processor-executable algorithm to select a design of the mechanism. In one example, data corresponding to motion occurring during actual surgical procedures is used to define both a dexterous workspace (DWS) and an extended dexterous workspace (EDWS). In one example, a method includes maximizing a measure of device ability to easily move in the DWS while still reaching all the EDWS called isotropy. In one example, parameters describing rotational inertia and the device stiffness are optimized. Other parameters or factors can be optimized including, for example, the sphere radius.

One example of this device includes two (or multiples of two) links to mechanically constrain two degrees of motion to the surface of a sphere. The motion in two degrees intersects or converges at one point defining a sphere. When a linear instrument is positioned at the end of the two (or multiples of two) links of such a device, the device constrains an axis of the instrument to intersect the center of the sphere for any configuration of the device. A minimally invasive surgical (MIS) instrument may be placed at the end of the two (or multiples of two) links and the rotation of said instrument will intersect the sphere. The device may be placed in a position where that instrument may function as normal but with a new, mechanically constrained and non-patient based, fulcrum of motion.

The sphere center can be placed in the trocar or at an arbitrary point internal or external to the patient's body. For example, when the sphere center is positioned outside the body, a mechanical support can be used to position the tool (between the base and sphere center) for use in open surgery.

The design of specific physical parameters used relevant data to optimize those parameters to maximize dexterity and minimize weight for the given purpose.

In the present subject matter, the rotation axes of two or more links are aligned to converge or intersect in a single point. At least one link or other structure is flexibly mounted and free to encircle the single point while traveling about the surface of a sphere centered on the single point. The single point, in various examples, is at or near the center of the mechanism.

In one example, the rotation axes intersect. In other examples, the rotation axes converge without intersecting, in which case, the discussion herein regarding point of intersection refers to a point at or near the minimum separation distance of the axes.

In the context of MIS, the point of intersection can be aligned with the location of the port through which a tool is inserted into the body. At the point of intersection, for example, movement of the tool is restricted to a generally conical volume with the vertex at the port. In addition, with a suitable joint, the tool is allowed to travel along an axis aligned with the point of intersection.

With respect to FIG. 11, center 40 of the sphere is the origin for the reference frames of the mechanism. Thus, each link frame is a pure rotation from one to the next.

The frames are assigned such that the z-axis of the n^{th} frame points outward along the n^{th} joint. The numbering scheme for the frames includes odd numbers (frames 0′, 1, 3 and 5). The end-effector, frame is frame 5. Frame 0′ is oriented such that the z-axis points along joint 1 and the y-axis points to the apex of the sphere.

The odd number subscript notations 0′, 1, 3, 5 of the present subject matter provides symmetry with an even side notation of 0″, 2, 4 and 6 of a parallel mechanism. The frames can be viewed as a shoulder, an elbow and a tool axis.

The link angle, αᵢ₊₂ expresses the angle between the i^{th} and (i+2)^{th} axis. These are fixed parameters defined by the mechanism geometry. The rotation angle θᵢ defines the angle as a function of time between the rotation axis i−2 and i. When all joint angles are set to zero (θ₁=θ₃=0), link α₁₃ lies in a plane defined by Z_{0′} and Y_{o′}, link α₃₅ is folded back on link α₁₃

Accordingly, FIG. 11 illustrates serial configured spherical motion mechanism 15A with coordinates Z₁, Z₃ and Z₅. Mechanism 15A includes base 50A coupled to link 60A at joint J1 at a first or base end. Axis Z₁ passes through revolute joint J1. In addition, second revolute joint J3 is located at a second end of link 60A. Axis Z₃ passes through revolute joint J3. Mechanism 15A also includes link 70A coupled to joint J3 at one end and having end 75A at a second end. Axis Z₅ passes through end 75A, and in various examples, includes a tool or an instrument having an axis aligned with center 40. In one example, end 75A includes a prismatic joint.

In various examples, the length, or angle of link 60A may be the same or different from the length, or angle of link 70A.

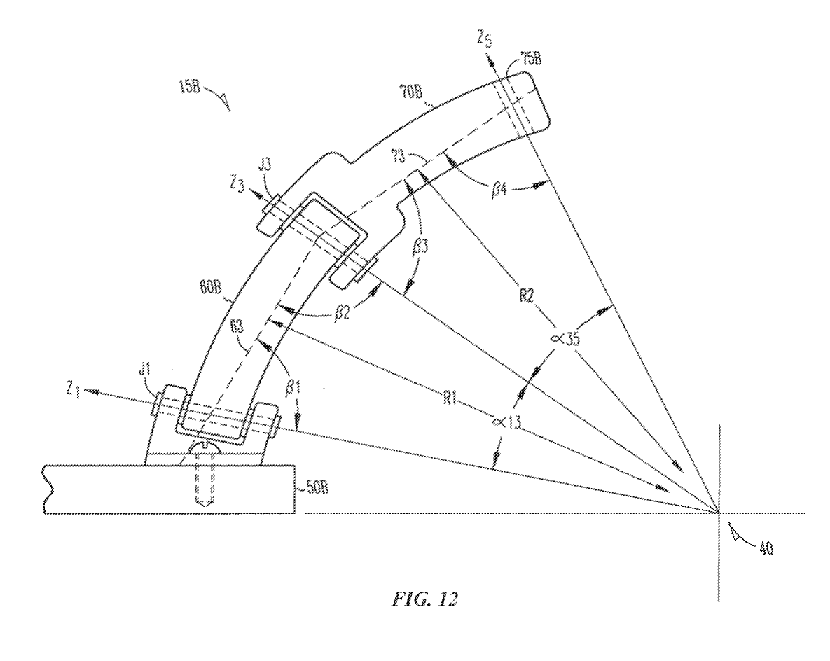

FIG. 12 illustrates a generalized view of spherical motion mechanism 15B. In the figure, base 50B is immobilized with respect to center 40, however, in other examples, base 50B is flexibly mounted. Joint J1 is affixed to base 50B by a threaded fastener and has axis Z₁ aligned with center 40. Dimension R1 is a measure of the length of the radius describing link 60B. As illustrated, linear length 63 passes through joint J1 and joint J3. In addition, dimension R2 is a measure of the length of the radius describing link 70B. As illustrated, linear length 73 passes through joint J3 and joint J5.

In the figure, interior angles β1, β2, β3 and β4 are all acute, however, in one example, at least one angle is 90 degrees.

The transformation matrices between frames are based on the Denavit-Hartenberg (DH) parameter notation and summarized in Table I. Since no translations exist between the assigned coordinate systems, the transformation matrix is reduced from the typical 4×4 matrix to a 3×3 rotation matrix.

TABLE I
Serial Manipulator D-H Parameters

| i − 1 | i | i + 1 | αᵢ₋₁ | θᵢ |
|---|---|---|---|---|
| 0′ | 1 | 3 | 0 | θ₁ |
| 1 | 3 | 5 | −α₁₃ | θ₃ |
| 3 | 5 | — | α₃₅ | −θ₅ = 0 |

As used herein, the term α refers to a “twist angle” in the DH notation and is considered an angular “link length.” The twist angle can be viewed as the “angle between axes of rotation” where the axes intersect at a point. In the DH notation, twist angle is defined as the angle between the axes measured about the common normal. Thus, for axes that tend to converge (such as two skew axes that are non-intersecting and non-parallel), the term α refers to a measure of an angle between the two axes about a line perpendicular (normal) to both axes.

Given the mechanism parameters (αᵢ₋₁, θᵢ) the forward kinematics aimed to express the orientation of the end-effector ^{0′} u expressed in frame 0′. Using the DH notation along with the DH parameters defined in Table I, the generalized rotation matrix is defined as follows:

$${}_{i}^{i - 1} R [ \begin{matrix}cos {θ}_{i} & - sin {θ}_{i} & 0 \\ sin {θ}_{i} * cos {α}_{i - 1} & cos {θ}_{i} * cos {α}_{i - 1} & - sin {α}_{i - 1} \\ sin {θ}_{i} * sin {α}_{i - 1} & cos {θ}_{i} * cos {α}_{i - 1} & cos {α}_{i - 1}\end{matrix} ] \qquad ( 11 )$$

The forward kinematics from the base, Frame 0′ to the end effector, Frame 5 is the product of those rotation matrices.

^{0′}₅R=^{0′}₁R*₃¹R*₅³R (12)

Rather than expressing the entire end-effector frame it is sensible to express a vector that represents the axis along which the surgical tool will point. In one example, tool roll θ₅ is designed onto the distal end of the mechanism. Let ^{0′} u be a vector pointing along the end-effector axis, z₅ expressed in Frame 0′.

$${ }^{{0}^{′}} u = {}_{5}^{{0}^{′}} R * [ \begin{matrix}0 \\ 0 \\ 1\end{matrix} ] = [ \begin{matrix}{ }^{{0}^{′}} {u}_{x} \\ { }^{{0}^{′}} {u}_{y} \\ { }^{{0}^{′}} {u}_{z}\end{matrix} ] = [ \begin{matrix}\begin{matrix}cos {θ}_{1} * sin {θ}_{3} * sin {α}_{35} + \\ sin {θ}_{1} * cos {θ}_{3} * cos {α}_{13} * sin {α}_{35} - \\ sin {θ}_{1} * sin {α}_{13} * cos {α}_{35}\end{matrix} \\ \begin{matrix}sin {θ}_{1} * sin {θ}_{3} * sin {α}_{35} - cos {θ}_{1} * \\ cos {θ}_{3} * cos {α}_{13} * sin {α}_{35} - \\ cos {θ}_{1} * sin {α}_{13} * cos {α}_{35}\end{matrix} \\ cos {θ}_{3} * sin {α}_{13} * sin {α}_{35} + cos {α}_{13} * cos {α}_{35}\end{matrix} ] \qquad ( 13 )$$

From the vector ^{0′}u that expresses the z-axis of the end-effector and the mechanism parameters (αᵢ₋₁), the inverse kinematics express the mechanism joint angles θᵢ. Using the expression for ^{0′}u_{z} (the third line of ^{0′}—Eq. 13) and solving for cos θ₃ results in

$$cos {θ}_{3} = \frac{{}_{}^{{0}^{′}}{u}{}_{z}^{} - cos {α}_{13} * cos {α}_{35}}{sin {α}_{13} * sin {α}_{35}} \qquad ( 14 )$$

Given the expression for cosine of θ₃ there are two possible solutions for θ₃, one corresponding to an elbow up and one for the elbow down configuration. The two solutions for θ₃ are as follows:

θ₃ₐ,θ_{3b}=a tan 2(±\sqrt{1−cos²θ₃}, cos θ₃) (15)

Using the expression for ^{0′}uₓ, ^{0′}u_{y} (the first and second lines of ^{0′}u—Eq. 13) and solving for sin θ₁ and cos θ₁ and finally θ₁ results in Eq. 16.

$$cos {θ}_{1} = \frac{{ }^{{0}^{′}} {u}_{x} * sin {θ}_{3} * sin {α}_{35} - {}_{}^{{0}^{′}}{u}{}_{y}^{} * ( \begin{matrix}cos {θ}_{3} * cos {α}_{13} * \\ sin {α}_{35} - sin {α}_{13} * cos {α}_{35}\end{matrix} )}{{( sin {θ}_{3} * sin {α}_{35} )}^{2} + {( \begin{matrix}cos {θ}_{3} * cos {α}_{13} * sin {α}_{35} - \\ sin {α}_{13} * cos {α}_{35}\end{matrix} )}^{2}} \\ sin {θ}_{1} = \frac{{ }^{{0}^{′}} {u}_{y} * sin {θ}_{3} * sin {α}_{35} {+}^{{0}^{′}} {u}_{x} * ( \begin{matrix}cos {θ}_{3} * cos {α}_{13} * \\ sin {α}_{35} - sin {α}_{13} * cos {α}_{35}\end{matrix} )}{{( sin {θ}_{3} * sin {α}_{35} )}^{2} + {( \begin{matrix}cos {θ}_{3} * cos {α}_{13} * sin {α}_{35} - \\ sin {α}_{13} * cos {α}_{35}\end{matrix} )}^{2}} \\ {θ}_{1} = a tan 2 ( sin {θ}_{1} , cos {θ}_{1} ) \qquad ( 16 )$$

Once the values for θ₃ corresponding to elbow up/down configurations are solved, the associated values for θ₁ can be solved using Eq. 16. Thus the inverse kinematic equations provide two solutions to the pose of the manipulator, θ₁ₐ and θ₃ₐ, and θ_{1b} and θ_{3b}.

The Jacobian matrix relates joint velocities to end-effector angular velocities. It can be expressed with respect to anyone of the frames associated with the mechanism. If the Jacobian is expressed in Frame 5, the eigenvalue corresponding to the angular velocity of Frame 5 has a value of 1 for all poses and joint velocities. This reduction in the Jacobian dimensions allows one to use the upper 2×2 submatrix of the 30 Jacobian. This truncated version of the Jacobian relates the two controlled joint velocities, 1 and 3 to end-effector velocity. In the most general sense a recursive expression of the angular velocity is expressed in Eq. 17.

[ⁱ⁺¹ωᵢ₊₁]=[₁ⁱ⁺¹R][ⁱωᵢ]+ [symbol] ᵢ₊₁¹⁾zᵢ₊₁ (17)

By distributing and rearranging, the expression from the end effector frame, Frame 5, to the base frame, Frame 0′ is stated as

$$[ {}_{}^{5}{ω}{}_{5}^{} ] = [ {}_{1}^{5} R ] [ \begin{matrix}0 \\ 0 \\ 1\end{matrix} ] + [ {}_{3}^{5} R ] [ \begin{matrix}0 \\ 0 \\ 1\end{matrix} ] {3}_{} + [ \begin{matrix}0 \\ 0 \\ 1\end{matrix} ] {5}_{} \qquad ( 18 )$$

which is translated into:

$$[ \begin{matrix}{}_{}^{5}{ω}{}_{5 x}^{} \\ {}_{}^{5}{ω}{}_{5 y}^{} \\ {}_{}^{5}{ω}{}_{5 z}^{}\end{matrix} ] = [ {}_{1}^{5} R ] [ \begin{matrix}0 \\ 0 \\ 1\end{matrix} ] [ {}_{3}^{5} R ] [ \begin{matrix}0 \\ 0 \\ 1\end{matrix} ] [ \begin{matrix}0 \\ 0 \\ 1\end{matrix} ] [ \begin{matrix}{1 -}_{} \\ {3}_{} \\ {5}_{}\end{matrix} ] \qquad ( 19 )$$

Based on the previous justification, the upper 2×2 submatrix of the full 3×3 Jacobian is taken resulting in a truncated 2×2 version relating the controlled axes of motion to the end effector velocity, expressed in Eq. 20.

$$[ \begin{matrix}{}_{}^{5}{ω}{}_{5 x}^{} \\ {}_{}^{5}{ω}{}_{5 xy}^{}\end{matrix} ] = {[ {J}_{5 truncated} ]}_{2 \times 2} [ \begin{matrix}{1}_{} \\ {3}_{}\end{matrix} ] \qquad ( 20 )$$

This version of the Jacobian is further used for calculating the manipulator isotropy, to be discussed elsewhere in this document.

In one example, data is collected corresponding to measurements of the position and orientation of two endoscopic tools along with the forces and torques applied to the tools in a minimally invasive environment. Analysis of a database of generic surgical tasks (including tissue handling/examination, tissue dissection, and suturing) performed on an animal model in-vivo by 30 surgeons in a MIS environment indicates that for 95% of the time, the positions of the surgical tools encompass a 60° cone with a tip located at the port. In addition, measuring the reachable workspace of an endoscopic tool performed on a human model showed that in order to reach the full extent of the abdomen the tool needed to move 90° in the lateral/medial direction (left to right) and 60° in the superior/inferior (foot to head) direction.

The reachable workspace of the spherical manipulator is a sector of a sphere. The size and the shape of this sector are determined by the mechanism link lengths (α₁₃, α₃₅) and joint limits. Based on the in-vivo measurements, for one example, the dexterous workspace (DWS) for the surgical robot was defined as the area on the sphere bounded by the closed line created when a right circular cone with a circular cross section and a vertex angle of 60° located at the center of the sphere intersected the sphere. The extended dexterous workspace (EDWS) of the surgical robot was defined in a similar manner, however, the ‘cone’ had an elliptical cross section created by two orthogonal vertex angles of 60° and 90°. The optimization process is directed to defining the mechanism parameters (link lengths) allowing it to reach the EDWS and provide high dexterity in the DWS.

The links of the present subject matter lie on the surface of a sphere of a particular radius. As such, their length can be measured by the angular extent between the two axes, one at each end and both intersecting or converging at the center of the sphere. Accordingly, link length refers to the angular extent between the two motion axes defining the link ends.

In one example, a compromise between the general objective to design a small form factor mechanism with a high dexterity workspace sufficiently large enough to reach the extended workspace as required in surgery was achieved by optimizing the mechanism link lengths to allow maximal dexterity in the DWS while including the EDWS in its reachable workspace.

Mathematically, the manipulator can move through singularities, fold on itself and overlap with external objects without regard to how a physical device might accomplish this.

Based on the mechanical design limits of the mechanism, in one example, the range of motion of the first joint angle is) 180° (0°<θ₁<180° and the range of motion of the second angle is 160° (20°<θ₃<180°. These constrains were further used to limit the space from which an optimal solution was searched.

The Jacobian matrix allows analysis of the kinematic performance of a mechanism. One performance metric using the Jacobian matrix is mechanism manipulability. This analysis uses mechanism isotropy as the performance metric. Isotropy is defined in Eq. 21 as the ratio between the lowest eigenvalue and the highest eigenvalue of the Jacobian.

$$ISO ( {θ}_{1} , {θ}_{3} ) = \frac{{λ}_{min}}{{λ}_{max}} ISO ∈ 〈 0 , 1 〉 \qquad ( 21 )$$

Given a design candidate (that is, a pair of link angles α₁₃ and α₃₅, for every given mechanism pose, the associated isotropy value is in the range of 0 to 1. An isotropy measure of 0 means the mechanism is in a singular configuration and has lost a degree of freedom. A measure of one means that the eigenvalues of the Jacobian are all equal and the mechanism can move equally well in all directions.

Once the kinematic equations and a performance measure are defined, one can take a design candidate with link angles α₁₃, α₃₅, and evaluate the performance at each point in mechanisms workspace. The integration of the isotropy over a DWS or EDWS is used as one of the parameters to define a scoring value for a specific mechanism candidate.

In one example, each design candidate (that is, a pair of link angles α₁₃ and α₃₅) is assigned a single score so the best overall manipulator design can be selected. Three individual criteria including: (1) an integrated average score; (2) a minimal single score; and (3) the cube of the angular length of the links are incorporated into the composite score and expressed in Eq. 23.

Mechanism isotropy is a performance measure for a particular pose of the manipulator. In order to analyze the mechanism, a hemisphere is discretized into points distributed equally in azimuth and elevation. This distribution causes each point to be associated with a different area based on elevation. One measure of how well a manipulator performs is to calculate the isotropy at each point, multiply by its corresponding area then sum all of the weighed point-scores over the sector generated when a cone with head angle of 60° and located at the center of the sphere intersects the reachable part of the hemisphere given the mechanism joint constraints previously defined. This score provides an average performance over the entire section intersected by the cone.

Given the ranges of the azimuth angle σ and the elevation angle ζ defining the intersection area between a right circular cross section cone with a vertex angle of 60° located at the center of the sphere and the sphere itself, the set of all possible intersection areas on the hemisphere is

K={k(σ,ζ):0<σ<2*π,0<ζ<π/4}

The set of all the discrete points contained in the intersection area is

k_{σ}^{P},ζ⊂k_{σ,ζ}

Due to the discrete nature of the computation, each point included in the intersection area has an associated isotropy value ISO and sector area A. Thus the overall scoring functions are

$$\begin{matrix}{S}_{sum} = {MAX}_{K} \{ {\sum }_{{k}_{σ}^{P} , ζ}^{ } ISO ( {θ}_{1} , {θ}_{3} ) * A ( σ , ζ ) \} & ( a ) \\ {S}_{min} = {MAX}_{K} \{ {MIN}_{{k}_{σ}^{P} , ζ} ( ISO ( {θ}_{1} , {θ}_{3} ) ) \} & ( b )\end{matrix}$$

There are a number of orientations of the DWS with respect to the hemisphere, noted as the set K. For each element of K, the summed isotropy is different. The value considered is the highest value of the summed isotropy score, which is further referred as Sₛᵤₘ. In other words, each design candidate has a Sₛᵤₘ value that corresponds to the highest summed isotropy score for that design.

The limitation of a summed isotropy score is that singularities or workspace boundaries could exist within a region that has a good score. The minimum isotropy value within the cone intersection area is an indicator of the worst performance that can be expected over that cone intersection area. For each element in K, the minimum isotropy is different. The value returned is the highest minimum isotropy score on the set of all cones, K, referred to as Sₘᵢₙ. In other words, each design candidate has a Sₘᵢₙ, value that corresponds to the best of all possible worst-case isotropy scores for that design.

A design with greater link angles will have a larger reachable workspace and generally better Sₛᵤₘ and Sₘᵢₙ values.

The drawback to larger link angles is a decrease in link stiffness and greater bulk. As suggested by the experimental findings, in surgery the mechanism is operated in a limited workspace. According to one example, the goal is to maximize the kinematic performance over the surgical workspace while minimizing the link length. Static analysis of a cantilever beam shows that the arm stiffness is inversely proportional to the cube of length.

The overall score for a design candidate with link lengths α₁₃ and α₃₅, taking into account all three criteria is defined as follows:

$$φ = \frac{{S}_{sum} \cdot {S}_{min}}{{( {α}_{13} + {α}_{35} )}^{2}} \qquad ( 23 )$$

According to one example, a requirement of the optimization is that over the DWS or EDWS, the mechanism does not encounter any singularities or workspace boundaries. By multiplying the summed isotropy by the minimum isotropy, candidates that fail to meet this requirement have a score of zero. By dividing by the cube of the sum of the link angles the score reflects proportionality to the mechanisms stiffness or mass. Thus, over a scan of the potential design space, the peak composite score represents a design with maximum average performance, a guaranteed minimum performance and maximized stiffness.

In one example, the optimization considered all combinations of α₁₃ and α₃₅ from 16° to 90° in 2° increments for a total of 1444 design candidates. The hemisphere was discretized into 3600 points, distributed evenly in azimuth and elevation.

$$min φ ( {α}_{13} , {α}_{35} , {θ}_{1} , {θ}_{3} ) \{ \begin{matrix}16 ° < {α}_{13} < 90 ° \\ 16 ° < {α}_{35} < 90 ° \\ {θ}_{1} ∈ DWS \\ {θ}_{3} ∈ DWS\end{matrix} \qquad ( 24 )$$

Considering the DWS, its orientation in azimuth and elevation were varied in order to obtain the best cone for that design candidate. However, optimizing the EDWS, which is an elliptical cone, would add another design parameter, namely cone roll angle. Introducing an additional parameter will increase execution time of the optimization by an order of magnitude. By utilizing a 90° cone that encompasses the EDWS, a superset of the EDWS was created which eliminates the additional design parameter. However, using a superset of the EDWS could force the link lengths to be larger than necessary. For example a design that can reach 60° in one direction and 90° in an orthogonal direction satisfies the EDWS cone but not a 90° cone.

Using the definition of the scoring criteria a numerical scan of the design space was performed using all the combinations of link angles α₁₃ and α₃₅ in the range of 16° to 90°. Optimizing on the DWS, the best design was achieved with link angles of α₁₃=52° and α₃₅=40° and a score of 0.0520. In contrast, running the same optimization but requiring a 90° cone indicated that the optimal mechanism design has link angles α₁₃=90° and α₃₅=72° with a score of 0.0471.

The difference in the results is not unexpected but it does pose an interesting dilemma. If one chooses the design that optimizes on a 90° cone, the resulting design should be more likely to reach all the poses that manipulator would be asked to reach. However, this design has lower overall performance than the design optimized for the DWS and larger links, which may increase the likelihood for problems of collisions between two manipulators.

One interesting consideration is to take the best design that is optimized for the DWS that also has the ability to reach a 90° cone. This is done by taking the set of designs from the 90°-cone optimization with a non-zero score (these are all designs which have some 90° cone that contains no singularities) and run an optimization on this subset of designs. Effectively it takes the DWS optimization and slices out the designs that cannot reach a 90° cone.

The resulting peak in the design space is α₁₃=74° and α₃₅=60° with a score of 0.0367. This design is a compromise of the DWS optimization and the 90° cone optimization. However as discussed earlier, optimization on a 90° cone may result in a design that is larger than needed. The workspace of the optimal design for the DWS (α₁₃=52° and α₃₅=40° is a slice of a sphere.

The foregoing describes development of the kinematic equations for the serial spherical manipulator with arbitrary link angles. Optimization of the mechanism specifically for surgery yields a more compact device than a general spherical manipulator. The optimization balanced between a guaranteed minimum and integrated isotropy over the DWS as well as total link length in order to yield a very compact, high-dexterity mechanism.

Other design and performance parameters can also be evaluated. For example, a metric corresponding to dynamic performance can be selected. As another example, optimal placement of two or more manipulators over a patient can be determined based on parameters such as robot patient collisions as well as robot-robot collisions and self-collision.

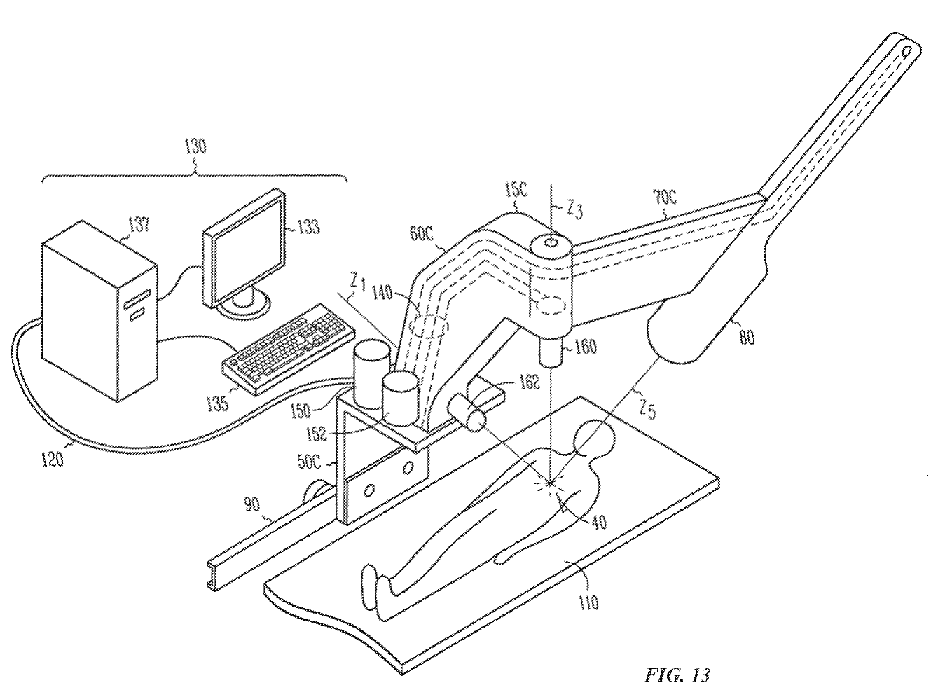

FIG. 13 illustrates spherical motion mechanism 15C affixed to track 90 aligned with platform 110. Track 90 allows base 50C to travel along the length of a patient on platform 110. Base 50C also provides mounting for drive motors 150 and 152, each of which are coupled to particular elements of mechanism 15C by cable actuators 140 routed within the structure of link 60C and link 70C. The routing of cable actuators 140 is configured such that the cable remains taut and direction changes are aligned about axes Z₁, Z₃ and Z₅. In addition, the figure illustrates light sources 160 and 162 also aligned with axes Z₁ and Z₃. An additional light source (not shown) can be aligned with axis Z₅. Light sources 160 and 162, in one example, include laser diodes configured to project a pinpoint light source at center 40. According to one example, adjusters on base 50C allow an operator to align mechanism 15C to co-locate center 40 with a trocar of the patient.

In one example, the cable actuators are routed over pulleys configured on bearings that are aligned with the revolute joints. In the example illustrated, the drive motors are mounted on the stationary base 50C, thus reducing the mass articulated on the revolute joints. In one example, a helical groove on the motor shaft engages a cable routed through the mechanism. The cable is coupled to a linear bearing which carries an instrument or tool aligned with axis Z₅.

In one example, a light source generates a pattern that facilitates alignment of the axes with a trocar or other target location. Such patterns include, for example, a bull's-eye pattern, a cross-hair pattern concentric circles or other such shapes to facilitate alignment. The wall thickness or irregularities in the body position can blur the alignment and a cross-hair pattern, for example, allows alignment without regard to wall thickness. Light sources 160 and 162, in one example, include threaded fasteners that align with the axes of the revolute joints.

Mechanism 15C is coupled to computer 130 by connecting line 120. In one example, computer 130 includes processor 137, user input device 135 such as a keyboard, mouse or other controller, and display 133 or printer. Processor 137 includes a memory and is configured to execute a set of instructions to perform a method as described elsewhere in this document.

Instrument 80, in the example illustrated includes a tool affixed to a prismatic joint. In one example, instrument 80 includes an optical instrument or other device aligned with center 40 through axis Z₅. Motion of instrument 80 can include travel along axis Z₅ or rotation about axis Z₅.

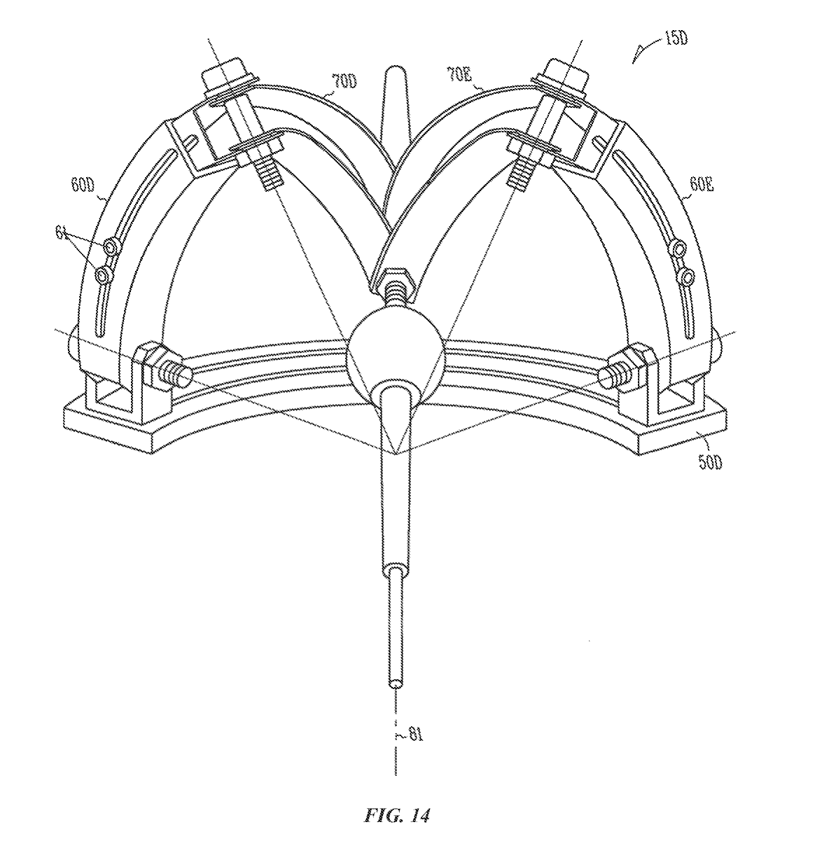

FIG. 14 illustrates a view of an exemplary parallel spherical motion mechanism 15D mounted on base 50D. Base 50D includes a curved structure having slotted holes to receive base joints coupled to link 60D and link 60E. In the example illustrated, link 60D and link 60E have adjustable length and are secured by locking screws 61. For example, by reducing the overall length of link 60D and link 60E, the mechanism is reduced in size and provides motion throughout a smaller cone. In the example illustrated, a change in the length of a link will change the angle of the axes of the revolute joints. Link 70D and 70E terminate at a common revolute joint through which tool axis 81 is aligned. In other examples, a spherical motion mechanism according to the present subject matter includes a link having a fixed (non-adjustable) length or angle.

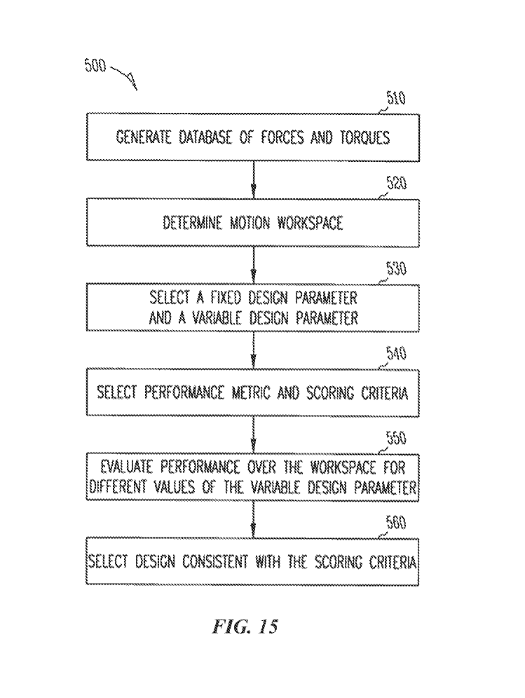

FIG. 15 includes flowchart 500 that describes a design method corresponding to the present subject matter. The method can be implemented by a processor executing a set of instructions, an analog computer or other machine. For example, at 510, a database of forces, torque and other measured date is generated. In the foregoing example, data was collected based on measured parameters during surgical procedures with a number of physicians.

At 520, the motion workspace is determined. In the example, the dexterous workspace and the extended dexterous workspace was defined in terms of a mathematical relationship.

At 530, the fixed design parameters and variable design parameters are selected. The parameters-can include such values as the length of each linkage element, the overall radius between the center point and the linkage as well as the angle corresponding to the revolute joints and the center point. Other parameters can also be selected.

At 540, at least one performance metric or scoring criteria is selected for use in comparing the various designs generated. In the example above, the designs were evaluated based on a ratio tailored to heavily penalize designs having long linkage elements. The design preference, according to this metric, reflects favoring a shorter and more compact structure.

At 550, the method includes evaluating the different designs for performance over the workspace using different values of the variable design parameter. In one example, the workspace is evaluated with a granularity of 2 degrees.

At 560, a target design is selected based on the scoring criteria. In various examples, the selection process may include minimizing, maximizing or meeting a particular numeric value.

Other methods are also contemplated for selecting a particular design. For example, one method provides maintaining a constant radius and selecting angles based on a desired range of motion. Other methods entail selecting both a radius and an angle or other combinations to provide the desired motion.

In addition to the aforementioned tools or instruments, the present subject matter can be configured for use with optical devices such as a light source, a minor or a camera. Audio devices such as a microphone or sound generator or ultrasonic transducer are also contemplated. In one example, a manipulator device is used. A representative manipulator device includes a grasper, a paddle, a spatula, or a stent delivery device. Furthermore, the tool can include a biopsy probe or other device for extracting a sample or a drug delivery device. An electrical tool can include a cauterizing tool or a sensor such as a pressure or temperature sensor.

The figures illustrate serial and parallel structures, however, other combinations are also contemplated. For example, one embodiment includes three sets of links configured in a parallel manner to constrain motion about a sphere. As such, the motion of the tool is limited to travel along or about an axis through the center of the sphere.

The joint axes of the present subject matter converge to the center of a sphere. In contrast, the joint axes of a planar machine are generally parallel and the links lie in a plane.

In one example, a first spherical motion mechanism and a second spherical motion mechanism are affixed to a common base and have end effectors linked by a linear member. In one example, the range of motion of a revolute joint is limited, for instance, by a mechanical stop.

The end effector, in various examples, includes a tool receiver configured to receive one or more of a variety of tools or instruments. Each tool or instrument can have, for example, a prismatic joint or other combination of up to three independent axis of motion.

In one example, the mechanism is powered by one or more electric motors. Other drivers are also contemplated, including, for example, manual motion, hydraulic or pneumatic actuators.

### CONCLUSION

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

What is claimed is:

1. A device comprising:
a first link having ends terminated in a base revolute joint and a common revolute joint, the revolute joints having convergent rotational axes and each rotational axis forming an acute angle with a longitudinal axis of the first link, the base revolute joint coupled to a base;
a second link coupled to the common revolute joint at a first end, the second link having a second end and the second link in a serial cantilever configuration with the first link, the rotational axis of the common revolute joint forming an acute angle with a longitudinal axis of the second link, wherein the second end of the second link includes a tool holder, the tool holder having a tool axis aligned to pass through a point coincident with an intersection of the convergent rotational axes, the tool axis and the common revolute joint rotational axis subtending a first angle; and
the convergent rotational axes subtending a second angle, such that the first angle differs from the second angle, the first and second links and the revolute joints enabling a position of the tool holder to be selectively manipulated.

2. The device of claim 1 wherein the convergent rotational axes subtends an angle of less than 90 degrees.

3. The device of claim 1 wherein the first angle is smaller than the second angle.

4. The device of claim 1 further including a light source coupled proximate to at least one revolute joint, wherein illumination from the light source is directed toward an intersection of the convergent rotational axes, such that each such light source and the tool holder are adjacent.

5. The device of claim 1 wherein the intersection of the convergent rotational axes comprises a first point and wherein the tool holder has a tool axis aligned to pass through a second point, the first point being different than the second point.

6. The device of claim 1 wherein an amount that at least one of the links extends along its longitudinal dimension is adjustable.

7. The device of claim 1 wherein each link comprises a first arcuate member and a second arcuate member disposed in a parallel facing relationship.

8. The device of claim 1 wherein a workspace achieved by the device is controlled by the relative dimensions of the first link and the second link, and the dimensions of the first link and second link have been selected to achieve a workspace corresponding to an inverted cone having a vertex disposed at a point at which the convergent axes intersect, and having a maximum vertex angle of 90 degrees or less.

9. The device of claim 8 wherein the inverted cone has a maximum vertex angle of 60 degrees or less.

10. The device of claim 8 wherein the inverted cone has a first vertex angle of about 60 degrees and a second vertex angle of about 90 degrees.

11. The device of claim 1 wherein the first angle is about 72 degrees and the second angle is about 90 degrees.

12. The device of claim 1 wherein the first angle is about 40 degrees and the second angle is about 52 degrees.

13. The device of claim 1 wherein the first angle is about 60 degrees and the second angle is about 74 degrees.

14. The device of claim 1 further comprising a first cable actuator coupling a motor to the tool holder, the first cable actuator being routed along the first link such that the first cable actuator provides motion to a tool in the tool holder.

15. The device of claim 14 wherein the first cable actuator is routed over a pulley having an axis aligned with the common revolute joint.

16. The device of claim 14 further including a second motor coupled to the base and coupled to at least one of the base revolute joint and the common revolute joint by a second cable actuator.

17. The device of claim 14 wherein the tool holder is configured to receive a tool and wherein the first cable actuator is configured to manipulate the tool to provide at least one of rotary motion on a tool axis and prismatic motion on the tool axis.

18. A method comprising:
providing a first joint coupled to a base and having a first rotational axis;
pivotally coupling a first link to the first joint at a first end wherein the first rotational axis and a first longitudinal axis of the first link form an acute angle, the first link having a second end;
forming a second joint having a second rotational axis at the second end, wherein the second rotational axis and the first longitudinal axis form an acute angle, the first and second rotational axes converging at a common point, thereby defining a first link angle; and
coupling a second link to the second joint at a first end of the second link, the second link and the first link in a serial cantilever configuration and the second link having a second longitudinal axis wherein the second longitudinal axis and the second rotational axis form an acute angle, the second link having an instrument receiver disposed at a distal end, the distal end opposite the first end of the second link.

19. The method of claim 18 further including affixing a light source to at least one of the first joint and the second joint.

20. The method of claim 18 further including coupling a cable actuator to the first link.

21. The method of claim 18 further including coupling a cable actuator to the second link.

22. The method of claim 18 further including coupling an electric control to at least one of the first joint and the second joint.

23. The method of claim 18 further comprising the steps of:
coupling a first cable actuator to the instrument receiver, the first cable actuator coupled to a first motor and the first motor coupled to the base; and
coupling a second cable actuator to at least one of the first joint and second joint, the second cable actuator coupled to a second motor and the second motor coupled to the base.

24. A system including:
a subject platform;
an alignment mechanism coupled to the subject platform, the alignment mechanism having a first link coupled to a base by a first revolute joint and a second link coupled to the first link by a second revolute joint, wherein the revolute joints have convergent rotational axes that subtend an acute angle and wherein the second link includes an instrument holder the first link and the second link being in a serial cantilever configuration, the instrument holder coupled to a cable actuator, the cable actuator coupled to a motor and the motor coupled to the base, the instrument holder having an instrument axis aligned to pass through a point coincident with an intersection of the convergent rotational axes, the instrument axis and the rotational axis of the first revolute joint defining a first link angle, the convergent rotational axes of the first revolute joint and the second revolute joint defining a second link angle, such that the first link angle differs from the second link angle, the first and second links and the first and second revolute joints enabling a position of the instrument holder to be selectively manipulated;
a driver coupled to the alignment mechanism and configured to control a position of at least one of the first link and the second link; and
a controller coupled to the driver and coupled to the motor.

25. The system of claim 24 wherein the instrument holder is configured to receive an optical element, a probe, a sensor or a manipulator device.

26. The system of claim 24 wherein the driver includes an electric motor affixed to the base.

27. The system of claim 24 wherein the driver includes a cable and pulley coupled to the at least one of the first link and the second link.

28. The system of claim 24 further including a light coupled to at least one of the first revolute joint and the second revolute joint.

29. The system of claim 24 wherein the instrument holder includes a linear actuator.

30. The system of claim 24 further including a second alignment mechanism coupled to the subject platform, the second alignment mechanism having a first link and a second link coupled to a revolute joint wherein an end of the second link is configured to travel in a sphere and wherein the second link of the second alignment mechanism is coupled to the instrument holder, each alignment mechanism being coupled to a common base, such that the different alignment mechanisms are coupled to spaced apart locations on the common base.

31. The system of claim 24 further including:
a processor configured to communicate with the driver; and
a user control communicatively coupled to the processor and configured to receive a user input for control of the alignment mechanism.

32. A spherical motion mechanism for controlling a motion of a tool in a workspace, the spherical motion mechanism comprising:
a first link having ends terminated in a base revolute joint and a common revolute joint, the revolute joints having convergent rotational axes and each rotational axis forming an acute angle with a longitudinal axis of the first link, the base revolute joint being coupled to a base;
a second link coupled to the common revolute joint at a first end, the second link having a second end and the second link in a serial cantilever configuration with the first link, the rotational axis of the common revolute joint forming an acute angle with a longitudinal axis of the second link, wherein the second end of the second link includes a tool holder, the tool holder having a tool axis aligned to pass through a point coincident with an intersection of the convergent rotational axes; wherein
a size of the workspace achieved by the spherical motion mechanism is controlled by relative dimensions of the first link and the second link, and the dimensions of the first link and second link have been selected to achieve a workspace corresponding to an inverted cone having a vertex disposed at a point at which the convergent axes intersect, and having a maximum vertex angle of 90 degrees or less.

33. A parallel spherical motion mechanism for controlling a motion of a tool in a workspace, the parallel spherical motion mechanism comprising:
a supporting base including a first attachment point and a second attachment point, the first and second attachment points being spaced apart;
a first spherical motion mechanism comprising
a first link having ends terminated in a first base revolute joint and a first common revolute joint, the first revolute joints having convergent rotational axes and each rotational axis forming an acute angle with a longitudinal axis of the first link, the first base revolute joint being coupled to the first attachment point of the base; and
a second link coupled to the first common revolute joint at a first end, the second link having a second end and the second link in a serial cantilever configuration with the first link, the rotational axis of the first common revolute joint forming an acute angle with a longitudinal axis of the second link;
a second spherical motion mechanism comprising:
a third link having ends terminated in a second base revolute joint and a second common revolute joint, the second revolute joints having convergent rotational axes and each rotational axis forming an acute angle with a longitudinal axis of the third link, the second base revolute joint being coupled to the second attachment point of the base; and
a fourth link coupled to the second common revolute joint at a first end, the fourth link having a second end and the fourth link being in a serial cantilever configuration with the third link, the rotational axis of the second common revolute joint forming an acute angle with a longitudinal axis of the fourth link; and
a tool holder supportively coupled to the second end of the second link and the second end of the fourth link, the tool holder having a tool axis aligned to pass through a point coincident with an intersection of each of the convergent rotational axes, the first and second attachment points being equidistant from the intersection of the convergent rotational axes.

34. The parallel spherical motion mechanism of claim 33 wherein a dimension of each of the first, second, third, and fourth links is adjustable.
